(19) United States
(12) Patent Application Publication
(10) Pub. No.: US 2016/0341745 A1
(43) Pub. Date: Nov. 24, 2016

(54) Methods, Systems, and Composition Related to Neural Disorders

(71) Applicant: AMARANTUS BIOSCIENCE HOLDINGS, INC., San Francisco, CA (US)

(72) Inventors: Gerald COMMISSIONG, Hummelstown, PA (US); John COMMISSIONG, Sunnyvale, CA (US); David A. LOWE, Vevey (CH)

(21) Appl. No.: 14/976,646

(22) Filed: Dec. 21, 2015

Related U.S. Application Data
(63) Continuation of application No. PCT/US2014/043506, filed on Jun. 20, 2014.
(60) Provisional application No. 61/837,452, filed on Jun. 20, 2013.

(51) Int. Cl. G01N 33/68; G06F 19/24; G06F 19/20

(52) U.S. Cl. CPC G01N 33/6896; G06F 19/20; G06F 19/24; G01N 2800/28; G01N 2800/50; G01N 2800/56

(57) ABSTRACT

The invention relates to methods for the diagnosis of CTE or the early stages thereof or a predisposition to CTE. The methods are based on quantitative determination of a mitogenically expressible surface markers, and peripherally accessible cells, e.g. skin cells or lymphocytes, (a) prior to and (b) after mitogenic stimulation. A specific stimulation index a:b is an indication of CTE or early stages thereof or of a predisposition to CTE. The invention also relates to kits which are suitable for carrying out the inventive methods of diagnosis.

### CROSS-REFERENCE

This application is a continuation of PCT/US2014/043506, filed Jun. 20, 2014, which claims priority to U.S. Provisional Application No. 61/837,452, filed Jun. 20, 2013, each of which application is incorporated herein by reference in their entirety.

### BACKGROUND OF THE INVENTION

The idea that repetitive blows to the head over a period of time, as occurs in boxers, resulting in traumatic brain injury (TBI), could produce a distinct brain pathology was first suggested in 1928, and called “dementia pugilistica” (Martland, 1928). Dementia pugilistica is now called chronic traumatic encephalopathy (CTE), or literally pathology in the head caused by chronic trauma to the brain. Clinical manifestations of TBI pathology include: 1) Memory disturbances, 2) Behavioral and personality changes, 3) Parkinsonism, 4) Speech and gait abnormalities, 5) Dementia and 6) Depression (McKee et al, 2009).

CTE is different from other diseases with similar symptoms (e.g., Alzheimer's Disease (AD)) as CTE can result from any physical activity in which the brain is repeatedly traumatized. For example, CTE and AD share similar, but distinct, pathologies. One is that in CTE, the oligomerized protein can be mainly tau (DeKosky et al, 2013). Nevertheless, amyloid neurofibrillary tangles that are primarily associated with AD do occur in some CTE cases (McKee et al, 2013). Brain shrinkage, expanded lateral ventricles and varying degrees of dementia can be common to both.

Football players, boxers, and military personnel exposed to loud, repetitive blasts can be prime candidates for CTE (Goldstein et al, 2012; McKee et al, 2013; Omalu et al, 2010). Not all boxers, or football players, or military personnel exposed to the same degree of brain trauma develop CTE (Jordan et al, 1997; Guskiewicz et al (2005). In one Canadian study 3 out of 6 retired football players with equivalent exposure to brain trauma did not develop CTE (Hazrati et al, 2013).

There are currently no consensus-based clinical diagnostic criteria for CTE. There is also no way to predict who, among the individuals exposed to trauma, will develop CTE.

Thus, the technical problem underlying the present invention is to provide an improved method for the reliable diagnosis of CTE, even allowing detecting early stages of the disease.

### SUMMARY OF THE INVENTION

Disclosed herein are methods, systems, kits, and compositions related to the detection and prediction of neural disorders related to trauma. In particular a diagnostic test for CTE, based on a reduced mitotic index for PBLs in CTE patients versus controls is disclosed.

In one aspect, disclosed herein are methods for diagnosing a subject with chronic traumatic encephalopathy, an early-stage of chronic traumatic encephalopathy, or a predisposition for chronic traumatic encephalopathy, the methods comprising: (a) preparing a stimulated sample by culturing a first portion of a biological sample obtained from the subject with one or more of mitogenic compounds and a reference sample by culturing a second portion of the biological sample without the one or more mitogenic compounds; (b) quantifying the expression of one or more surface markers in the stimulated sample and the reference sample; (c) normalizing the expression of the one or more surface markers in the stimulated sample with the expression of the one or more surface markers in the reference sample by determining one or more stimulation indices; and (d) relating the one or more stimulation indices to an assessment of the risk of developing chronic traumatic encephalopathy, a diagnosis of chronic traumatic encephalopathy, or a measure of the progression of chronic traumatic encephalopathy.

In some embodiments, the biological sample comprises a tissue sample, a blood sample, a bone marrow sample, a cerebrospinal fluid sample, or a combination thereof. In some embodiments, the biological sample comprises a blood sample. In some embodiments, the method further comprises isolating peripheral blood mononuclear cells (PMBCs) from the blood sample. In some embodiments, the stimulated sample and the reference sample are produced from the PMBCs.

In some embodiments, the method further comprises obtaining the biological sample from the subject.

In another aspect, disclosed herein are methods of diagnosing a subject with chronic traumatic encephalopathy, an early-stage of chronic traumatic encephalopathy, or a predisposition for chronic traumatic encephalopathy, the methods comprising: (a) isolating peripheral blood mononuclear cells (PMBCs) from a blood sample obtained from the subject; (b) culturing a first portion of the PMBCs with one or more mitogenic compounds to produce a stimulated sample; (c) culturing a second portion of the PMBCs without the one or more mitogenic compounds to produce a reference sample; (d) quantifying the expression of one or more surface markers in the stimulated sample and the reference sample; (e) normalizing the expression of the one or more surface markers in the stimulated sample with the expression of the one or more surface markers in the reference sample by determining one or more stimulation indices; and (f) relating the one or more stimulation indices to an assessment of the risk of developing chronic traumatic encephalopathy, a diagnosis of chronic traumatic encephalopathy, or a measure of the progression of chronic traumatic encephalopathy.

In some embodiments, the method further comprises obtaining the blood sample from the subject. In some embodiments, obtaining the blood sample comprises venous puncture.

In some embodiments, the normalizing is computer implemented.

In some embodiments, the relating is computer implemented.

In some embodiments, the method further comprises sending a result from the relating to a party via a communication medium.

In some embodiments, the one or more mitogenic compounds comprise phytohaemagglutinin (PHA-L), pokeweed mitogen (PWM), or a combination thereof.

In some embodiments, mitogenic compound is phytohaemagglutinin (PHA-L).

In some embodiments, the method further comprises preparing a second stimulated sample by culturing a third portion of the biological sample or the PMBCs with the mitogenic compound that is pokeweed mitogen (PWM). In some embodiments, the method further comprises quantifying the expression of the one or more surface markers in the second stimulated sample. In some embodiments, the method further comprises normalizing the expression of the one or more surface markers in the second stimulated sample with the expression of the one or more surface markers in the reference sample by determining one or more stimulation indices.

In some embodiments, quantifying the expression of the one or more surface markers comprises staining the stimulated sample and the reference sample with one or more antibodies that specifically bind the one or more surface markers. In some embodiments, the one or more antibodies are fluorescently labeled.

In some embodiments, quantifying the expression of the one or more surface markers comprises fluorescent activated cell sorting, western blotting, ELISA analysis, magnetic cell sorting, or a combination thereof. In some embodiments, quantifying the expression of the one or more surface markers comprises fluorescent activated cell sorting.

In some embodiments, the one or more surface markers comprise one or more cell type markers, one or more activation markers, or a combination thereof. In some embodiments, the one or more activation markers that are CD69, CD28, or a combination thereof. Some embodiments comprise the one or more cell type markers that are CD45, CD14, CD3, CD4, CD8, CD19, CD11b, CD114, CD15, CD24, CD182, CD11a, CD91, CD16, CD25, Foxp3, CD20, CD38, CD22, CD61, CD56, CD31, CD30, CD38, CD62L, CD127, CD132, CD45RA, CD45RO, CD34, CD31, CD117, CD44, or a combination thereof. In some embodiments, the one or more surface markers comprise CD45, CD14, CD3, CD4, CD8, CD19, CD69, CD28, or a combination thereof. In some embodiments, the one or more surface markers comprise CD45, CD14, CD3, CD4, CD8, CD19, CD69, and CD28.

In some embodiments, the one or more stimulation indices comprise a stimulation index 1 (SI1) defined by the ratio of a percentage of cells positive for one of the one or more surface markers with and without mitogenic stimulation within an analyzed cell population. In some embodiments, SI1 is calculated according to the following equation: SI1=(Marker⁺ Cells/Total Cells)ₛₜᵢₘ/(Marker⁺ Cells/Total Cells)ᵤₙₛₜᵢₘ.

In some embodiments, the one or more stimulation indices comprise a stimulation index 2 (SI2) defined by a ratio of mean expression for one of the one or more surface markers within an analyzed cell population with and without mitogenic stimulation. In some embodiments, SI2 is calculated according to the following equation: SI2=(Mean Marker Int.)stim/(Mean Marker Int.)unstim.

In some embodiments, the surface marker is an activation marker. In some embodiments, the surface marker is one or more activation markers comprising CD69, CD28, or a combination thereof.

In some embodiments, the analyzed cell population comprises total lymphocytes, T-lymphocytes, T helper/inducer lymphocytes, T suppressor/cytotoxic lymphocytes, monocytes, B lymphocytes, granulocytes, T regulatory cells, natural killer cells, thrombocytes, stem cells, naïve T lymphocytes, memory T lymphocytes, or a combination thereof.

In some embodiments, the analyzed cell population is identified by the expression of one or more surface markers. In some embodiments, the one or more surface markers are cell type markers comprising CD45, CD14, CD3, CD4, CD8, CD19, CD11b, CD114, CD15, CD24, CD182, CD11a, CD91, CD16, CD25, Foxp3, CD20, CD38, CD22, CD61, CD56, CD31, CD30, CD38, CD62L, CD127, CD132, CD45RA, CD45RO, CD34, CD31, CD117, CD44, or a combination thereof. In some embodiments, the analyzed cell population comprises total lymphocytes that are positive for expression of the surface marker CD45. In some embodiments, the analyzed cell population comprises T lymphocytes that are positive for expression of the surface marker CD3. In some embodiments, the analyzed cell population comprises T helper/inducer lymphocytes that are positive for the expression of the surface marker CD4. In some embodiments, the analyzed cell population comprises T suppressor/cytotoxic lymphocytes that are positive for the expression of the surface marker CD8. In some embodiments, the analyzed cell population comprises monocytes that are positive for the expression of the surface marker CD14, CD11a, CD91, CD16, CD114, CD11b, or a combination thereof. In some embodiments, the analyzed cell population comprises monocytes that are positive for the expression of the surface marker CD14. In some embodiments, the analyzed cell population comprises B lymphocytes that are positive for the expression of the surface marker CD19. In some embodiments, the analyzed cell population comprises B lymphocytes that are positive for the expression of the surface marker CD20+, CD24+, CD38, CD22, or a combination thereof. In some embodiments, the analyzed cell population comprises granulocytes that are positive for the expression of the surface marker CD15+, CD182+, CD11b, CD24+, CD114+, or a combination thereof. In some embodiments, the analyzed cell population comprises granulocytes that are positive for the expression of the surface marker CD15+, CD182+, or a combination thereof. In some embodiments, the analyzed cell population comprises T regulatory cells that are positive for the expression of the surface marker CD4, CD25, Foxp3, or a combination thereof. In some embodiments, the analyzed cell population comprises natural killer cells that are positive for the expression of the surface marker CD56, CD31, CD30, CD38, or a combination thereof. In some embodiments, the analyzed cell population comprises thrombocytes that are positive for the expression of the surface marker CD61. In some embodiments, the analyzed cell population comprises stem cells that are positive for the expression of the surface marker CD34, CD117, or a combination thereof. In some embodiments, the analyzed cell population comprises naïve T lymphocytes that are positive for the expression of the surface marker CD45RA, CD127, CD132, CD62L, or a combination thereof. In some embodiments, the analyzed cell population comprises memory T lymphocytes that are positive for the expression of the surface marker CD45RO.

In some embodiments, an SI1 an SI2 or both are determined based on the expression level of CD69 in one or more analyzed cell populations selected from the list comprising total lymphocytes, T-lymphocytes, T helper/inducer lymphocytes, T suppressor/cytotoxic lymphocytes, monocytes, B lymphocytes, granulocytes, T regulatory cells, natural killer cells, thrombocytes, stem cells, naïve T lymphocytes, and memory T lymphocytes.

In some embodiments, an SI1 an SI2 or both are determined, individually, based on the expression level of CD69 in each of one or more analyzed cell populations selected from the list comprising total lymphocytes, T-lymphocytes, T helper/inducer lymphocytes, T suppressor/cytotoxic lymphocytes, monocytes, B lymphocytes, granulocytes, T regulatory cells, natural killer cells, thrombocytes, stem cells, naïve T lymphocytes, and memory T lymphocytes.

In some embodiments, an SI1 an SI2 or both are determined, individually, based on the expression level of CD69 in each of two, three, four, five, six, seven, eight, nine, ten, eleven or twelve analyzed cell populations selected from the list comprising total lymphocytes, T-lymphocytes, T helper/inducer lymphocytes, T suppressor/cytotoxic lymphocytes, monocytes, B lymphocytes, granulocytes, T regulatory cells, natural killer cells, thrombocytes, stem cells, naïve T lymphocytes, and memory T lymphocytes.

In some embodiments, an SI1 an SI2 or both are determined based on the expression level of CD28 in each of one or more analyzed cell populations selected from the list comprising total lymphocytes, T-lymphocytes, T helper/inducer lymphocytes, T suppressor/cytotoxic lymphocytes, monocytes, B lymphocytes, granulocytes, T regulatory cells, natural killer cells, thrombocytes, stem cells, naïve T lymphocytes, and memory T lymphocytes.

In some embodiments, an SI1 an SI2 or both are determined based on the expression level of CD28 in each of two, three, four, five, six, seven, eight, nine, ten, eleven or twelve analyzed cell populations selected from the list comprising total lymphocytes, T-lymphocytes, T helper/inducer lymphocytes, T suppressor/cytotoxic lymphocytes, monocytes, B lymphocytes, granulocytes, T regulatory cells, natural killer cells, thrombocytes, stem cells, naïve T lymphocytes, and memory T lymphocytes.

In some embodiments, relating the one or more stimulation indices to an assessment of the risk of developing chronic traumatic encephalopathy, a diagnosis of chronic traumatic encephalopathy, or a measure of the progression of chronic traumatic encephalopathy comprises comparing one stimulation index to a univariate model that differentiates between two clinical categories. In some embodiments, relating comprises comparing the one or more stimulation indices to a multivariate model that differentiates between two clinical categories.

In some embodiments, the two clinical categories are chronic traumatic encephalopathy and healthy control, chronic traumatic encephalopathy and Alzheimer's Disease, chronic traumatic encephalopathy and Parkinson's disease, chronic traumatic encephalopathy and neural disorders that are not chronic traumatic encephalopathy, or chronic traumatic encephalopathy and not chronic traumatic encephalopathy. In some embodiments, the two clinical categories are chronic traumatic encephalopathy and healthy control. In some embodiments, the two clinical categories are chronic traumatic encephalopathy and Alzheimer's Disease. In some embodiments, the two clinical categories are chronic traumatic encephalopathy and Parkinson's disease. In some embodiments, the two clinical categories are chronic traumatic encephalopathy and neural disorders that are not chronic traumatic encephalopathy. In some embodiments, the two clinical categories are chronic traumatic encephalopathy and not chronic traumatic encephalopathy.

In some embodiments, the univariate model or multivariate model is capable of differentiating the two clinical categories with at least a 70%, 75%, 80%, 85%, 90%, 91%, 92%, 93%, 94%, 95%, 96%, 97%, 98%, or 99% positive agreement. In some embodiments, the univariate model or multivariate model is capable of differentiating the two clinical categories with at least a 70%, 75%, 80%, 85%, 90%, 91%, 92%, 93%, 94%, 95%, 96%, 97%, 98%, or 99% negative agreement.

In some embodiments, the subject is diagnosed with chronic traumatic encephalopathy based upon relating the one or more stimulation indices to an assessment of the risk of developing chronic traumatic encephalopathy, a diagnosis of chronic traumatic encephalopathy, or a measure of the progression of chronic traumatic encephalopathy.

In some embodiments, the subject is determined to have an increased risk of developing chronic traumatic encephalopathy based upon relating the one or more stimulation indices to an assessment of the risk of developing chronic traumatic encephalopathy, a diagnosis of chronic traumatic encephalopathy, or a measure of the progression of chronic traumatic encephalopathy.

In some embodiments, the subject is determined to have progressed to a more severe form of chronic traumatic encephalopathy based on relating the one or more stimulation indices to an assessment of the risk of developing chronic traumatic encephalopathy, a diagnosis of chronic traumatic encephalopathy, or a measure of the progression of chronic traumatic encephalopathy.

In some embodiments, the method further comprises quantifying the ratio of memory T lymphocytes to naïve T lymphocytes by quantifying the expression level of CD45RO and CD45RA and determining a change in CD45RO/RA ratio between the stimulated sample and the unstimulated sample.

In some embodiments, the method further comprises administration of MANF or a MANF peptidergic molecule that crosses the blood brain barrier to the subject that is diagnosed with chronic traumatic encephalopathy.

In some embodiments, the method further comprises administration of CDNF or a CDNF peptidergic molecule that crosses the blood brain barrier to the subject that is diagnosed with chronic traumatic encephalopathy.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates exemplary gating of FACS data from the analysis of unstimulated peripheral blood mononuclear cells stained with an isotype cocktail of antibodies to cell markers including α-CD3-FITC, α-IGG1-PE, α-CD14-PerCP-Cy5.5, α-CD4-PE-CY7, α-IGG1-APC, α-CD45-APC-H7, α-CD19-V450, and α-CD8-V500 antibodies.

FIG. 2 illustrates exemplary gating of FACS data from the analysis of unstimulated peripheral blood mononuclear cells stained with a test cocktail of antibodies to cell and activation markers including α-CD3-FITC, α-CD69-PE, α-CD14-PerCP-Cy5.5, α-CD4-PE-CY7, α-CD28-APC, α-CD45-APC-H7, α-CD19-V450, and α-CD8-V500 antibodies.

FIG. 3 illustrates exemplary gating of FACS data from the analysis of peripheral blood mononuclear cells stimulated with PWM and stained with a test cocktail of antibodies to cell and activation markers including α-CD3-FITC, α-CD69-PE, α-CD14-PerCP-Cy5.5, α-CD4-PE-CY7, α-CD28-APC, α-CD45-APC-H7, α-CD19-V450, and α-CD8-V500 antibodies.

FIG. 4 illustrates an exemplary courses of events related to a method of diagnosing chronic traumatic encephalopathy.

FIG. 5 depicts a computer system useful for displaying, storing, retrieving, or calculating diagnostic results from a level of one or more surface markers; displaying, storing, retrieving, or calculating raw data from surface markers; or displaying, storing, retrieving, or calculating any sample or subject information useful in the diagnostic methods disclosed herein.

### DETAILED DESCRIPTION OF THE INVENTION

### Definitions

As used herein the singular forms “a”, “an” and “the” include plural references unless the context clearly dictates otherwise. For example, the term “a sample” includes a plurality of samples, including mixtures thereof.

As used herein the term “about” means the indicated numerical value±10%. All numerical indications used herein are to be interpreted as being qualified by “about” unless the context clearly indicates otherwise.

As used herein the term “or” can be used conjunctively or disconjunctively.

As used herein open terms, for example, “contain”, “containing”, “include”, “including”, and the like mean comprising unless otherwise indicates.

As used herein the term “diagnose” or “diagnosis” of a condition includes predicting or diagnosing the condition, determining predisposition to the condition, monitoring treatment of the condition, diagnosing a therapeutic response of the disease, and prognosis of the condition, condition progression, and response to particular treatment of the condition.

As used herein the term “sample” can mean or refer to any biological sample, including tissue and cellular samples.

As used herein, the term “marker” can mean or refer to any biological molecule (or fragment thereof) of interest, e.g., a biomarker, which is present on the cell surface.

As used herein, the term “cell staining” can mean or refer to the use of a reagent to enable the marker to be detected, for instance by binding to the marker and providing a detectable signal.

The present invention relates to a method of diagnosing neural disorders related to trauma or an early stage of or predisposition for these disorders.

Trauma can include physical insult to the head. Trauma can include neuronal injuries, hemorrhages, vascular injuries, cranial nerve injuries, and subdural hygromas, among many other types of injuries. In some instances the trauma does not induce any clinically observable symptoms at the time of insult. Trauma can include multiple instances of trauma. Trauma can be concussive or blast energy. Trauma can be the result of contact sports.

In some instances trauma will result in concussion. In some instances the trauma is less serious than a concussion. In some instances, the trauma does not result in a concussion.

Trauma, in some instances, can be the result of high acceleration of the brain or repeated exposure to high acceleration (G-force). Trauma, in some instances, can be the result of high deceleration or repeated exposure to high deceleration.

The trauma may be known. In other instances the trauma is unknown. In some instances the trauma can be measured, for example with an accelerometer or using a calculation such as the head injury criterion.

### Neural Disorders

The methods, compositions, kits, and systems disclosed herein can be used to aid in the detection (e.g., diagnosis, early detection, or detection of a predisposition) of neural disorders associated with trauma.

The disorder being detected can be defined by phenotype. The phenotype may be one or more of memory disturbances, behavioral and personality changes, parkinsonism, speech and gait abnormalities, dementia, depression, impulsive behavior, post traumatic stress disorder (PTSD), and/or substance abuse. For example the methods used herein could predict a person's predisposition to certain trauma induced phenotypic changes induced by trauma.

The disorder being detected can be defined by physical brain abnormalities. For example, a test described herein may be able to predict the likelihood that a person has developed, or is likely to develop, physical brain abnormalities; for example enlarged brain ventricles or cavum septum pellucidum.

The disorder being detected can be defined by post-mortem analysis. For example, a test described herein may be able to predict the likelihood that, upon post-mortem analysis, certain markers are found; for example, oligomerized and/or hyperphosphorylated tau protein or amyloid neurofibrillary tangles.

The disorder being detected can be a progressive degenerative disease. The disorder being detected can be an encephalopathy. The disorder being detected can be chronic traumatic encephalopathy (CTE).

### Timing of the Detection

The detection can occur prior to exposure to trauma. For example, a soldier may be tested for susceptibility for neural disorders associated with trauma before that soldier is assigned to a duty with a high likelihood of trauma exposure.

The detection can occur prior to exposure to trauma in order to establish a baseline for comparison. For example, a subject (e.g., a soldier, athlete, etc.) can be tested prior to engaging in an activity with a high likelihood of exposure to trauma, and later testing can determine deviation from the initial baseline. A baseline can also be re-established following a period of time away from the activity with a high likelihood of exposure to trauma.

The detection can also occur after exposure to trauma. For example a football player may be tested using the methods described herein after years of exposure to trauma in high school and college football, but before entering the NFL. Such testing may allow recommendations as to the risk involved with further exposure.

The detection can occur after exposure to trauma but before the onset of symptoms. Early detection of neural disorders associated with trauma may provide for early intervention and more productive therapy.

### Samples

The detection can comprise analysis of a sample. The analysis can occur in a sample obtained from an individual. The sample can contain cells, e.g. mitogenically stimulable cells. Suitable samples can include dermal tissue samples, blood samples, venous blood samples, Cerebrospinal fluid (CSF), or cells from the urine. When a blood sample is used, an anticoagulative compound, e.g. sodium citrate or heparin, can be added for the purpose of stabilization prior to the other method steps. When blood is used, peripheral blood mononuclear cells can be isolated from the blood and used in the methods disclosed herein.

The sample can include peripherally accessible cells, e.g. cells which can be removed without an operation or in a (minimally) invasive fashion from a patient. The peripherally accessible cells can be e.g. skin cells and peripheral blood mononuclear cells (e.g., lymphocytes) of the peripheral blood.

Cells in the sample can be kept alive and cultured. For example peripheral blood mononuclear cells (PBMC) can be isolated and cultured in media.

Cells in the sample can be stimulated prior to or during culturing. For example isolated cells can be stimulated with phytohaemagglutinine (e.g. PHA-L, 12 ug/ml, Sigma Aldrich St. Louis, Mo., USA) and/or Pokeweed mitogen (e.g. PWM, 4 μg/ml, Sigma Aldrich). The cells can be stimulated by PHA, protein A and/or PWM. Reference samples can be non-stimulated. The stimulated and/or reference samples can be frozen and stored.

The stimulation can be mitogenic stimulation. The mitogenic stimulation for obtaining the expression of surface markers can be effected by known stimulators, such as phytohemagglutinin (PHA), protein A, PWM or other compounds having a trophic or mitogenic effect. Such stimulators are referred to as mitogenic compounds. The stimulation can be effected by adding the individual mitogenic compounds or by a combined addition. Other mitogenic compounds that can be used in the methods disclosed herein can include nerve growth factor (NGF), fibroblast growth factor (FGF2), concanavalin A (conA), lipopolysaccharide (LPS), or any combinations thereof.

The concentration of a mitogenic compound can be within a certain physiological range depending upon, e.g., the type of mitogenic compound used and the condition under which the mitogenic stimulation is carried out. For example, the concentration of PHA may be from about 1 μg/mL to about 20 μg/mL, the concentration of PWM may be from about 1 μg/mL to about 50 μg/mL, the concentration of Protein A may be from about 10 μg/mL to about 200 μg/mL, the concentration of NGF may be from about 20 ng/mL to about 200 ng/mL, the concentration of FGF2 may be from about 1 ng/mL to about 20 ng/mL, the concentration of conA may be from about 10 μg/mL to about 1000 μg/mL, the concentration of LPS may be from about 10 μg/mL to about 1000 μg/mL.

The mitogenic compound can be added for a defined time period, depending upon the expression rate of the molecule to be examined. For example, an amount of time for stimulation can be from about 2 to about 72 hours. In some examples, an amount of time for mitogenic stimulation may be from about 2 to about 48 hours. In some examples, an amount of time for mitogenic stimulation may be from about 2 to about 24 hours. In some examples, an amount of time for mitogenic stimulation may be less than or equal to about 24 hours, less than or equal to about 20 hours, less than or equal to about 16 hours, less than or equal to about 12 hours, less than or equal to about 8 hours, less than or equal to about 7 hours, less than or equal to about 6 hours, less than or equal to about 5 hours, less than or equal to about 4 hours, less than or equal to about 3 hours, less than or equal to about 2 hours, or less than or equal to about 1 hour.

Suitable experimental conditions for mitogenic stimulation, e.g. as regards the concentration of the mitogenic compound used, the duration of stimulation and other incubation conditions can be used. The stimulation should be carried out in suitable vessels permitting adequate gas exchange. The concentrations of the respective stimulation agents should be within the physiological range which is 1 μg/ml to 20 μg/ml for PHA, 1 μg/ml to 50 μg/ml for PWM, and 10 μg/ml to 200 μg/ml for protein A. The stimulation period depends on the expression rate of the molecule to be examined. However, in some embodiments the stimulation periods are between 2 to 24 hours. In one example, for CD69, a stimulation period of about 4 hours can be used. Stimulation can be carried out under physiological conditions and it can be conducted in a gassing incubator at 37° C. and with 5% CO2, for example.

### Sample Analysis

Biomarkers in the samples can be analyzed. For example surface markers on the cells from the sample can be analyzed using, e.g., fluorescence activated cell sorting (FACS) and antibodies (e.g., monoclonal antibodies, polyclonal antibodies, or fragments thereof). Surface markers can include cell type markers, which can be used to identify subpopulations of cells in a sample. Surface markers can also include activation markers, which can be used to detect mitogenic stimulation or activation of cells in a sample. Cell type markers can include CD45, CD14, CD3, CD4, CD8, CD19, CD11b, CD114, CD15, CD24, CD182, CD11a, CD91, CD16, CD25, Foxp3, CD20, CD38, CD22, CD61, CD56, CD31, CD30, CD38, CD62L, CD127, CD132, CD45RA, CD45RO, CD34, CD31, CD117, CD44, or a combination thereof. Activation markers can include CD69, CD28, CD45RO, CD63, CD62L, or any combination thereof. Combinations of cell type markers and activation markers can be used to detect mitogenic stimulation or activation in subpopulations of cells within the sample. Some surface markers can act as both a cell type marker and an activation marker. For example, CD45RO and CD45RA can be used to identify memory and naïve T lymphocytes respectively, and a change in the ratio of CD45RO/RA before and after mitogenic stimulation can indicate a change in mitogenic activity.

Some examples of surface markers by means of which a mitogenic stimulation manifests itself include activation markers such as CD69, CD25, CD45RO, CD63 and HLA-Dr. For the purposes of the invention, it is also possible to carry out a determination of a combination of surface markers or the further specification of the cells separated by means of a certain surface marker, e.g., CD69, as regards further sub-populations of peripheral blood mononuclear cells (PBMCs) (e.g., peripheral blood lymphocytes (PBSs)), e.g., by means of cell type markers (e.g. CD45 and/or CD14 and/or CD3 and/or CD4 and/or CD8 and/or CD19 and/or CD11b and/or CD114 and/or CD15 and/or CD24 and/or CD182 and/or CD11a and/or CD91 and/or CD16 and/or CD25 and/or Foxp3 and/or CD20 and/or CD38 and/or CD22 and/or CD61 and/or CD56 and/or CD31 and/or CD30 and/or CD38 and/or CD62L and/or CD127 and/or CD132 and/or CD45RA and/or CD45RO and/or CD34 and/or CD31 and/or CD117 and/or CD4) subpopulations. These subpopulations can be referred to as the analyzed cell population.

In some instances, the analyzed cell population comprises total lymphocytes that are positive for expression of the surface marker CD45. In some instances, the analyzed cell population comprises T lymphocytes that are positive for expression of the surface marker CD3. In some instances, the analyzed cell population comprises T helper/inducer lymphocytes that are positive for the expression of the surface marker CD4. In some instances, the analyzed cell population comprises T suppressor/cytotoxic lymphocytes that are positive for the expression of the surface marker CD8. In some instances, the analyzed cell population comprises monocytes that are positive for the expression of the surface marker CD14, CD11a, CD91, CD16, CD114, CD11b, or a combination thereof. In some instances, the analyzed cell population comprises monocytes that are positive for the expression of the surface marker CD14. In some instances, the analyzed cell population comprises B lymphocytes that are positive for the expression of the surface marker CD19. In some instances, the analyzed cell population comprises B lymphocytes that are positive for the expression of the surface marker CD20+, CD24+, CD38, CD22, or a combination thereof. In some instances, the analyzed cell population comprises granulocytes that are positive for the expression of the surface marker CD15+, CD182+, CD11b, CD24+, CD114+, or a combination thereof. In some instances, the analyzed cell population comprises granulocytes that are positive for the expression of the surface marker CD15+, CD182+, or a combination thereof. In some instances, the analyzed cell population comprises T regulatory cells that are positive for the expression of the surface marker CD4, CD25, Foxp3, or a combination thereof. In some instances, the analyzed cell population comprises natural killer cells that are positive for the expression of the surface marker CD56, CD31, CD30, CD38, or a combination thereof. In some instances, the analyzed cell population comprises thrombocytes that are positive for the expression of the surface marker CD61. In some instances, the analyzed cell population comprises stem cells that are positive for the expression of the surface marker CD34, CD117, or a combination thereof. In some instances, the analyzed cell population comprises naïve T lymphocytes that are positive for the expression of the surface marker CD45RA, CD127, CD132, CD62L, or a combination thereof. In some instances, the analyzed cell population comprises memory T lymphocytes that are positive for the expression of the surface marker CD45RO. In any of these subpopulations, the expression level of an activation marker (e.g., CD69, CD28, CD45RO, CD63, CD62L) can be analyzed on a per-cell basis (e.g., % of cells positive for the activation marker) or on a population level (e.g., mean expression level within the population).

Antibodies can be used in the detection and/or quantitation of expression levels of surface markers. The term “antibody” as used herein relates to antibodies comprising polyclonal antibodies, pooled monoclonal antibodies with different epitopic specificities, as well as distinct monoclonal antibody preparations. Monoclonal antibodies can be made from an antigen containing fragments of the hAβ1-42 peptide and hAβ1-40, respectively by methods well known to those skilled in the art. As used herein, the term “antibody” (Ab) or “monoclonal antibody” (Mab) is meant to include intact molecules as well as antibody fragments (such as, for example, Fab and F(ab′)2 fragments) which are capable of specifically binding to protein. Fab and F(ab′)2 fragments lack the Fc fragment of intact antibody. Moreover, these include chimeric and single chain antibodies.

Antibodies can be detectably labeled, for example, with a radioisotope, a bioluminescent compound, a chemiluminescent compound, a fluorescent label, a metal chelate, or an enzyme (e.g. horse-radish peroxidase, alkaline phosphatase, β-galactosidase, malate dehydrogenase, glucose oxidase, urease, catalase etc.) which, in turn, when later exposed to a substrate will react to the substrate in such a manner as to produce a chemical moiety which can be detected. The probes can also be immobilized on an insoluble carrier, e.g. glass, polystyrene, polypropylene, polyethylene, dextran, nylon, natural and modified celluloses, polyacrylamides, agarose and magnetic beads.

Fluorescent labels that can be used include, but are not limited to, fluorescein, fluorescein isothiocyanate (FITC), tetramethyl rhodamine isothiocyanate (TRITC), rhodamine, tetramethyl rhodamine, R-phycoerythrin phycoerythrin, phycocyanin, allophycocyanin (APC), Cy-2, Cy-3, Cy-3.5, Cy-5, Cy5.5, Cy-7, Sybr Green I, Sybr Green II, Sybr Gold and Lissamine, eosin, green fluorescent protein, erythrosin, coumarin, methyl coumarin, pyrene, malachite green, stilbene, lucifer yellow, cascade blue, carboxy tetrachloro fluorescein, 5 and/or 6-carboxy fluorescein (FAM), 5-(or 6-) iodoacetamidofluorescein, 5-{[2(and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), and/or derivatives of any one or more of the above. In some instances, fluorescent labels can be conjugated to the antibodies.

Cells of the sample (e.g., PMBCs isolated from a blood sample) can be stained with antibodies to surface markers. The antibodies can include antibodies that specifically bind to surface markers such as CD45, CD14, CD3, CD4, CD8, CD19, CD11b, CD114, CD15, CD24, CD182, CD11a, CD91, CD16, CD25, Foxp3, CD20, CD38, CD22, CD61, CD56, CD31, CD30, CD38, CD62L, CD127, CD132, CD45RA, CD45RO, CD34, CD31, CD117, CD44, CD69, CD28, CD63, or any combination thereof. As discussed, the antibodies can be detectably labeled, e.g., fluorescently labeled.

Cells of the sample (e.g., PMBCs isolated from a blood sample) can be stained with one or more antibody cocktails. For example, cells can be stained with an isotype cocktail comprising α-CD3-FITC, α-IGG1-PE, α-CD14-PerCP-Cy5.5, α-CD4-PE-CY7, α-IGG1-APC, α-CD45-APC-H7, α-CD19-V450, and α-CD8-V500 antibodies. Alternatively, or in addition, cells can be stained with a test cocktail comprising α-CD3-FITC, α-CD69-PE, α-CD14-PerCP-Cy5.5, α-CD4-PE-CY7, α-CD28-APC, α-CD45-APC-H7, α-CD19-V450, and α-CD8-V500 antibodies.

Cells of the sample can be stained with one or more of the following antibodies tagged with a fluorescent marker: A—IgG1-FITC/IgG1-PE/CD45-PerCP-Cy5.5/IgG1-APC, B—CD8-FITC/CD69-PE/CD3-PerCP-Cy5.5/CD4-APC, C—CD14-FITC/CD69-PE/CD45-PerCP-Cy5.5/CD19-APC, D—CD8-FITC/CD28-PE/CD45-PerCP-Cy5.5/CD4-APC, E—CD45RA-FITC/CD45RO-PE/CD3-PerCP-Cy5.5/CD4-APC.

The expression level of one or more surface markers in a sample can be quantitated. For example, expression levels can be quantitated on a cell-by-cell basis using flow cytometry (e.g., FACS).

Raw data determined by measuring expression levels of surface markers can be reviewed, analyzed and processed. The information pertaining to the diagnosis of the disease can be obtained based upon the raw data and/or processed data. The obtained information may be provided to a recipient.

Surface marker expression data between stimulated and unstimulated samples can be self-normalized, for example, to account for differences in basal mitogenic activity between subjects. Self normalization can comprise calculation of one or more stimulation indices. Self-normalization can be accomplished by determining a stimulation index 1 (SI1) defined by the ratio of the percentages of cells positive for an surface marker (e.g., a cell type marker or an activation marker), with and without mitogenic stimulation, within an analyzed cell population (e.g., within a population identified by one or more cell type markers). Self-normalization can also be accomplished by determining a stimulation index 2 (SI2) defined by the ratio of surface marker expression level (e.g., a cell type marker or an activation marker) within an analyzed cell population (e.g., within a population identified by one or more cell type markers) with and without mitogenic stimulation. Each stimulation index therefore represents an analysis of a surface marker (e.g., activation marker), in a cell population (e.g., as determined by one or more cell type markers) as a result of treatment with a mitogenic compound (e.g., PHA-L, PWM, etc.).

The formulas for SI1 and SI2 are as follows:

$$SI 1 = \frac{( {Marker}^{+} Cells / Total Cells ) stim}{( {Marker}^{+} Cells / Total Cells ) unstim}$$

$$SI 2 = \frac{( Mean Marker Int . ) stim}{( Mean Marker Int . ) unstim}$$

The stimulation index (activation index) follows from the relationship of the number of cells bearing the surface marker or markers, or the average expression level of the surface marker or markers within an analyzed cell population, before and after the stimulation. A stimulation index which reaches at least 10 times, as a maximum 100 times, the unstimulated control sample, can be a sign of a CTE or an early stage of or a predisposition for this disease. A stimulation index which is less than 10 times the unstimulated control sample can indicate that the subject does not have CTE or an early stage of or a predisposition for this disease. The cells bearing the surface markers can be determined according to conventional methods, e.g. Western blot, ELISA, RIA, FACS, LSC, etc.

In order to determine the cells bearing the surface markers, they can be separated from the cells bearing no surface marker or bearing other surface markers by means of characteristic cell features.

In the diagnostic method of the present invention, the cells bearing the surface markers can be separated from the cells which bear no surface markers by antibodies directed against the desired surface marker(s). The antibodies suited for this purpose may be monoclonal, polyclonal or synthetic antibodies or fragments thereof. In this connection, the term “fragment” means all the parts of the monoclonal antibody (e.g., Fab, Fv or single chain Fv fragments) which have an epitope specificity the same as that of the complete antibody.

In some embodiments the antibody or antibodies specific to surface markers are bound to magnetic particles, e.g. paramagnetic beads (e.g., available from DYNAL A.S., P.O. Box 158 Skoyen, N-0212 Oslo, Norway), which permits the separation of the cells with the corresponding surface markers via immunomagnetic separation.

The stimulation index can then be specified by determining the amount of cells separated by means of the desired surface marker on the basis of its nucleic acid content and/or protein content using current methods, e.g., after lysis of the cells and spectrophotometric determination of the nucleic acid or protein content or after staining the nucleic acid using specific dyes, e.g. ethidium bromide, propidium iodide, acridine orange, DAPI, etc., by means of photometric quantification. The cell number can be calculated from the protein and/or nucleic acid content of the sample by means of standard calibration curves.

In some embodiments, CD45 isoform alteration in CD4+ T cells can be a diagnostic marker of CTE. CD45RA, CD45RO, CD45RB isoforms on CD4+ T cells sub-populations can be accessed via Flow Cytometry. CTE patients may have lower numbers of naïve CD4+ T cells as measured by CD45RA expression level. Without being bound by theory, although this result will not be a functional cell cycle result, it will indicate a weakened mitogenic response capability in CTE patients in the CD4+ lymphocyte sub-populations.

Cells of the sample can be exposed to Rapamycin to investigate cell cycle abnormalities. For example, Rapamycin can be used on peripheral blood lymphocytes in suspected CTE cases. Rapamycin based assessment of G1/S checkpoint integrity may indicate peripheral regulatory dysfunctions measurable in the blood lymphocytes of CTE patients compared to healthy controls. Without being bound by theory it is believed that if the cells of the sample have good G1/S check point regulatory function, then use of a G1/S inhibitor should lengthen the time that lymphocytes were staying in the G1 phase because they were being blocked from advancing to S phase by the Rapamycin present in the assay. On the other hand, if a CTE patient's lymphocytes had a faulty cell cycle or G1/S check point, then Rapamycin should not significantly elongate the time that Rapamycin treated lymphocytes from CTE patients remain in the G1 phase before moving through a leaky or faulty G1/S check point. Accordingly, in some embodiments, cells of the sample are exposed to Rapamycin to assess CTE. For example, CTE may be indicated when the percentage of cells in G1 phase goes down in the sample patients whereas the S phase percentage goes up.

In certain embodiments, a neural disorder associated with trauma (e.g., CTE) can be diagnosed based upon a stimulation index. The stimulation index can be the ratio of a cell surface marker (e.g., as determined by FACS using any of the antibodies disclosed herein) between stimulated and unstimulated samples containing PBSs. As already described, the stimulated samples can be produced by mitogenic stimulation. The stimulation index, as determined in the subject, can then be compared to a stimulation index from a control population or to a reference stimulation index value (collectively, control stimulation index). The reference stimulation index can be a baseline stimulation index determined in the subject prior to engaging in activity prone to trauma. The reference stimulation index can be determined from a control population comprising subjects that do not have the neural disorder associated with trauma. The subject can be diagnosed with the neural disorder associated with trauma if the stimulation index indicates mitotic disregulation. Mitotic disregulation can be indicated by a stimulation index that is lower than the control stimulation index. Mitotic disregulation can be indicated by a stimulation index that is higher than the control stimulation index.

For example, the subject can be diagnosed with the neural disorder associated with trauma based upon a reduced stimulation index in comparison to the control stimulation index. In some embodiments, a stimulation index that is reduced by from about 5% to about 95% in comparison to the control stimulation index can indicate that the subject has the neural disorder associated with trauma. For example, the subject can be diagnosed with the neural disorder associated with trauma if the stimulation index is about 5-95%, 5-75%, 5-50%, 5-25%, 5-15%, 5-10%, 10-75%, 10-50%, 10-25%, 10-15%, 15-75%, 15-50%, 15-25%, 25-75%, 25-50%, 50-75%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, or 95% lower than the control stimulation index. In another embodiment, the subject is diagnosed with the neural disorder associated with trauma if the stimulation index is at least 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, or 95% lower than the control stimulation index.

In another example, the subject can be diagnosed with the neural disorder associated with trauma based upon an increased stimulation index in comparison to the control stimulation index. In some embodiments, a stimulation index that is increased by from about 5% to about 100% in comparison to the control stimulation index can indicate that the subject has the neural disorder associated with trauma. For example, the subject can be diagnosed with the neural disorder associated with trauma if the stimulation index is about 5-95%, 5-75%, 5-50%, 5-25%, 5-15%, 5-10%, 10-75%, 10-50%, 10-25%, 10-15%, 15-75%, 15-50%, 15-25%, 25-75%, 25-50%, 50-75%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, or 95% higher than the control stimulation index. In another embodiment, the subject is diagnosed with the neural disorder associated with trauma if the stimulation index is at least 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, or 95% higher than the control stimulation index.

In another example, the subject can be diagnosed with the neural disorder associated with trauma based upon an increased stimulation index in comparison to the control stimulation index. In some embodiments, a stimulation index that is increased by from about 0.05 fold to about 10 fold in comparison to the control stimulation index can indicate that the subject has the neural disorder associated with trauma. For example, the subject can be diagnosed with the neural disorder associated with trauma if the stimulation index is about 0.05-10×, 0.05-5×, 0.05-2.5×, 0.05-1×, 0.05-0.5×, 0.05-0.25×, 0.05-0.1×, 0.1-10×, 0.1-5×, 0.1-2.5×, 0.1-1 X, 0.1-0.5×, 0.1-0.25×, 0.25-10×, 0.25-5×, 0.25-2.5×, 0.25-1×, 0.25-0.5×, 0.5-10×, 0.5-5 X, 0.5-2.5×, 0.5-1×, 1-10×, 1-5×, 1-2.5×, 2.5-10×, 2.5-5×, 5-10×, 0.05×, 0.06×, 0.07 X, 0.08×, 0.09×, 0.1×, 0.11×, 0.12×, 0.13×, 0.14×, 0.15×, 0.16×, 0.17×, 0.18×, 0.19 X, 0.2×, 0.21×, 0.22×, 0.23×, 0.24×, 0.25×, 0.26×, 0.27×, 0.28×, 0.29×, 0.3×, 0.31×, 0.32×, 0.33×, 0.34×, 0.35×, 0.36×, 0.37×, 0.38×, 0.39×, 0.4×, 0.41×, 0.42×, 0.43×, 0.44×, 0.45×, 0.46×, 0.47×, 0.48×, 0.49×, 0.5×, 0.6×, 0.7×, 0.8×, 0.9×, 1×, 1.1×, 1.2×, 1.3×, 1.4×, 1.5×, 1.6×, 1.7×, 1.8×, 1.9×, 2×, 2.1×, 2.2×, 2.3×, 2.4×, 2.5×, 2.75×, 3×, 3.25×, 3.5×, 3.75×, 4×, 4.25×, 4.5×, 4.75×, 5×, 5.5×, 6×, 6.5×, 7×, 7.5×, 8×, 8.5×, 9×, 9.5×, or 10×higher than the control stimulation index.

The subject can be diagnosed with the neural disorder associated with trauma based upon relating one or more stimulation indices to a diagnostic univariate model or a diagnostic multivariate model. The univariate or multivariate models can comprise data from subjects in one or more clinical categories. Exemplary clinical categories include, but are not limited to, healthy controls, subjects with a diagnosis or probable diagnosis of chronic traumatic encephalopathy, subject with Alzheimer's disease, subjects with Parkinson's disease, subject with neural disorders that are not chronic traumatic encephalopathy, or any combination thereof. The univariate or multivariate models can distinguish between any two of these clinical categories.

Diagnosis, according to the methods disclosed herein, can be based upon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 412, 42, 43, 44, 45, 46, 47, 48, or more variables (e.g., stimulation indices). As explained, a stimulation index can be based upon an analysis of a surface marker (e.g., activation marker), in a cell population (e.g., as determined by one or more cell type markers) as a result of treatment with a mitogenic compound (e.g., PHA-L, PWM, etc.). Therefore, a univariate or multivariate diagnostic model can be based upon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 412, 42, 43, 44, 45, 46, 47, 48, or more stimulation indices quantitated in subjects belonging to a clinical category. Table 1 (below) contains a non-exclusive list of stimulation indexes that can be included in a diagnostic model, thereby forming the basis for a diagnosis. It is contemplated that any and all combinations of the stimulation indices listed in Table 1 can be used in the diagnostic methods disclosed herein.

TABLE 1
exemplary stimulation indexes.

| Stimula- | Mitogenic | Activation | Cell Type | Analyzed Cell |
|---|---|---|---|---|
| tion Index | Compound | Marker | Marker | Population |
| SI1 | HLA | CD69 | CD45 | Total lymphocytes |
| SI1 | HLA | CD69 | CD3 | T lymphocytes |
| SI1 | HLA | CD69 | CD14 | Monocytes |
| SI1 | HLA | CD69 | CD4 | T-helper/inducer |
| | | | | lymphocytes |
| SI1 | HLA | CD69 | CD19 | B lymphocytes |
| SI1 | HLA | CD69 | CD8 | T suppressor/ |
| | | | | cytotoxic lymphocyte |
| SI1 | HLA | CD28 | CD45 | Total lymphocytes |
| SI1 | HLA | CD28 | CD3 | T lymphocytes |
| SI1 | HLA | CD28 | CD14 | Monocytes |
| SI1 | HLA | CD28 | CD4 | T-helper/inducer |
| | | | | lymphocytes |
| SI1 | HLA | CD28 | CD19 | B lymphocytes |
| SI1 | HLA | CD28 | CD8 | T suppressor/ |
| | | | | cytotoxic lymphocyte |
| SI1 | PWM | CD69 | CD45 | Total lymphocytes |
| SI1 | PWM | CD69 | CD3 | T lymphocytes |
| SI1 | PWM | CD69 | CD14 | Monocytes |
| SI1 | PWM | CD69 | CD4 | T-helper/inducer |
| | | | | lymphocytes |
| SI1 | PWM | CD69 | CD19 | B lymphocytes |
| SI1 | PWM | CD69 | CD8 | T suppressor/ |
| | | | | cytotoxic lymphocyte |
| SI1 | PWM | CD28 | CD45 | Total lymphocytes |
| SI1 | PWM | CD28 | CD3 | T lymphocytes |
| SI1 | PWM | CD28 | CD14 | Monocytes |
| SI1 | PWM | CD28 | CD4 | T-helper/inducer |
| | | | | lymphocytes |
| SI1 | PWM | CD28 | CD19 | B lymphocytes |
| SI1 | PWM | CD28 | CD8 | T suppressor/ |
| | | | | cytotoxic lymphocyte |
| SI2 | HLA | CD69 | CD45 | Total lymphocytes |
| SI2 | HLA | CD69 | CD3 | T lymphocytes |
| SI2 | HLA | CD69 | CD14 | Monocytes |
| SI2 | HLA | CD69 | CD4 | T-helper/inducer |
| | | | | lymphocytes |
| SI2 | HLA | CD69 | CD19 | B lymphocytes |
| SI2 | HLA | CD69 | CD8 | T suppressor/ |
| | | | | cytotoxic lymphocyte |
| SI2 | HLA | CD28 | CD45 | Total lymphocytes |
| SI2 | HLA | CD28 | CD3 | T lymphocytes |
| SI2 | HLA | CD28 | CD14 | Monocytes |
| SI2 | HLA | CD28 | CD4 | T-helper/inducer |
| | | | | lymphocytes |
| SI2 | HLA | CD28 | CD19 | B lymphocytes |
| SI2 | HLA | CD28 | CD8 | T suppressor/ |
| | | | | cytotoxic lymphocyte |
| SI2 | PWM | CD69 | CD45 | Total lymphocytes |
| SI2 | PWM | CD69 | CD3 | T lymphocytes |
| SI2 | PWM | CD69 | CD14 | Monocytes |
| SI2 | PWM | CD69 | CD4 | T-helper/inducer |
| | | | | lymphocytes |
| SI2 | PWM | CD69 | CD19 | B lymphocytes |
| SI2 | PWM | CD69 | CD8 | T suppressor/ |
| | | | | cytotoxic lymphocyte |
| SI2 | PWM | CD28 | CD45 | Total lymphocytes |
| SI2 | PWM | CD28 | CD3 | T lymphocytes |
| SI2 | PWM | CD28 | CD14 | Monocytes |
| SI2 | PWM | CD28 | CD4 | T-helper/inducer |
| | | | | lymphocytes |
| SI2 | PWM | CD28 | CD19 | B lymphocytes |
| SI2 | PWM | CD28 | CD8 | T suppressor/ |
| | | | | cytotoxic lymphocyte |

### Exemplary Embodiments

Disclosed herein are methods, systems, kits, and compositions related to the detection and prediction of neural disorders related to trauma. In particular a diagnostic test for CTE, based on a reduced mitotic index for PBLs in CTE patients versus controls is disclosed.

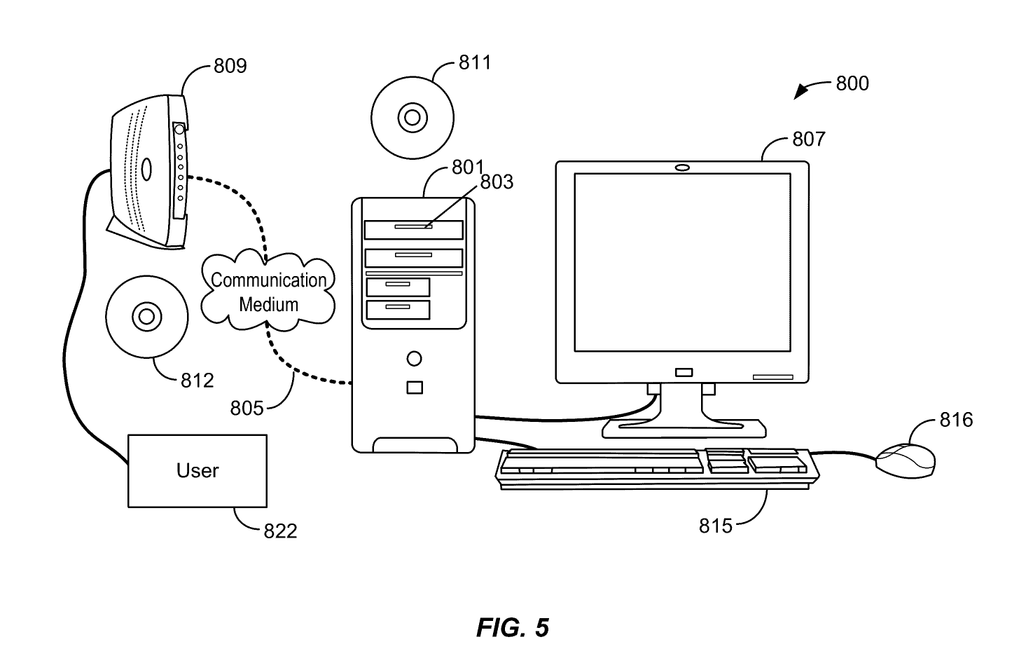

FIG. 5 illustrates an exemplary practice of the diagnostic methods disclosed herein. A sample can be collected from a subject. The method of collecting the biological sample can depend upon the type of biological sample collected. For example, where the biological sample is a blood sample, the sample can be collected by venous puncture. A first portion of the biological sample is cultured with a mitogenic compound to produce a stimulated sample. A second portion of the biological sample is cultured under the same conductions but without the mitogenic compound to produce a reference sample. The expression levels of one or more surface markers is quantified, for example, by FACS analysis. The expression levels are normalized between the stimulated sample and the unstimulated sample to account for differences is basal mitogenic activities between subjects. Normalization can be accomplished by calculating stimulation indices, as described herein. The stimulation indices are then related to a diagnostic model (e.g., a univariate or multivariate model) to determine a diagnosis for the subject. The normalization and/or relating functions can be computer implemented. The resulting diagnosis can be sent to a party via a communication media. Based on the results of the diagnosis, the patient can be treated for chronic traumatic encephalopathy.

In one aspect, disclosed herein are methods for diagnosing a subject with chronic traumatic encephalopathy, an early-stage of chronic traumatic encephalopathy, or a predisposition for chronic traumatic encephalopathy, the methods comprising: (a) preparing a stimulated sample by culturing a first portion of a biological sample obtained from the subject with one or more of mitogenic compounds and a reference sample by culturing a second portion of the biological sample without the one or more mitogenic compounds; (b) quantifying the expression of one or more surface markers in the stimulated sample and the reference sample; (c) normalizing the expression of the one or more surface markers in the stimulated sample with the expression of the one or more surface markers in the reference sample by determining one or more stimulation indices; and (d) relating the one or more stimulation indices to an assessment of the risk of developing chronic traumatic encephalopathy, a diagnosis of chronic traumatic encephalopathy, or a measure of the progression of chronic traumatic encephalopathy.

In some instances, the biological sample comprises a tissue sample, a blood sample, a bone marrow sample, a cerebrospinal fluid sample, or a combination thereof. In some instances, the biological sample comprises a blood sample. In some instances, the method further comprises isolating peripheral blood mononuclear cells (PMBCs) from the blood sample. In some instances, the stimulated sample and the reference sample are produced from the PMBCs.

In some instances, the method further comprises obtaining the biological sample from the subject.

In another aspect, disclosed herein are methods of diagnosing a subject with chronic traumatic encephalopathy, an early-stage of chronic traumatic encephalopathy, or a predisposition for chronic traumatic encephalopathy, the methods comprising: (a) isolating peripheral blood mononuclear cells (PMBCs) from a blood sample obtained from the subject; (b) culturing a first portion of the PMBCs with one or more mitogenic compounds to produce a stimulated sample; (c) culturing a second portion of the PMBCs without the one or more mitogenic compounds to produce a reference sample; (d) quantifying the expression of one or more surface markers in the stimulated sample and the reference sample; (e) normalizing the expression of the one or more surface markers in the stimulated sample with the expression of the one or more surface markers in the reference sample by determining one or more stimulation indices; and (f) relating the one or more stimulation indices to an assessment of the risk of developing chronic traumatic encephalopathy, a diagnosis of chronic traumatic encephalopathy, or a measure of the progression of chronic traumatic encephalopathy.

In some instances, the method further comprises obtaining the blood sample from the subject. In some instances, obtaining the blood sample comprises venous puncture.

In some instances, the normalizing is computer implemented.

In some instances, the relating is computer implemented.

In some instances, the method further comprises sending a result from the relating to a party via a communication medium.

In some instances, the one or more mitogenic compounds comprise phytohaemagglutinin (PHA-L), pokeweed mitogen (PWM), or a combination thereof.

In some instances, mitogenic compound is phytohaemagglutinin (PHA-L).

In some instances, the method further comprises preparing a second stimulated sample by culturing a third portion of the biological sample or the PMBCs with the mitogenic compound that is pokeweed mitogen (PWM). In some instances, the method further comprises quantifying the expression of the one or more surface markers in the second stimulated sample. In some instances, the method further comprises normalizing the expression of the one or more surface markers in the second stimulated sample with the expression of the one or more surface markers in the reference sample by determining one or more stimulation indices.

In some instances, quantifying the expression of the one or more surface markers comprises staining the stimulated sample and the reference sample with one or more antibodies that specifically bind the one or more surface markers. In some instances, the one or more antibodies are fluorescently labeled.

In some instances, quantifying the expression of the one or more surface markers comprises fluorescent activated cell sorting, western blotting, ELISA analysis, magnetic cell sorting, or a combination thereof. In some instances, quantifying the expression of the one or more surface markers comprises fluorescent activated cell sorting.

In some instances, the one or more surface markers comprise one or more cell type markers, one or more activation markers, or a combination thereof. In some instances, the one or more activation markers that are CD69, CD28, or a combination thereof. Some instances comprise the one or more cell type markers that are CD45, CD14, CD3, CD4, CD8, CD19, CD11b, CD114, CD15, CD24, CD182, CD11a, CD91, CD16, CD25, Foxp3, CD20, CD38, CD22, CD61, CD56, CD31, CD30, CD38, CD62L, CD127, CD132, CD45RA, CD45RO, CD34, CD31, CD117, CD44, or a combination thereof. In some instances, the one or more surface markers comprise CD45, CD14, CD3, CD4, CD8, CD19, CD69, CD28, or a combination thereof. In some instances, the one or more surface markers comprise CD45, CD14, CD3, CD4, CD8, CD19, CD69, and CD28.

In some instances, the one or more stimulation indices comprise a stimulation index 1 (SI1) defined by the ratio of a percentage of cells positive for one of the one or more surface markers with and without mitogenic stimulation within an analyzed cell population. In some instances, SI1 is calculated according to the following equation: SI1=(Marker⁺ Cells/Total Cells)ₛₜᵢₘ/(Marker⁺ Cells/Total Cells)ᵤₙₛₜᵢₘ.

In some instances, the one or more stimulation indices comprise a stimulation index 2 (SI2) defined by a ratio of mean expression for one of the one or more surface markers within an analyzed cell population with and without mitogenic stimulation. In some instances, SI2 is calculated according to the following equation: SI2=(Mean Marker Int.)stim/(Mean Marker Int.)unstim.

In some instances, the surface marker is an activation marker. In some instances, the surface marker is one or more activation markers comprising CD69, CD28, or a combination thereof.

In some instances, the analyzed cell population comprises total lymphocytes, T-lymphocytes, T helper/inducer lymphocytes, T suppressor/cytotoxic lymphocytes, monocytes, B lymphocytes, granulocytes, T regulatory cells, natural killer cells, thrombocytes, stem cells, naïve T lymphocytes, memory T lymphocytes, or a combination thereof.

In some instances, the analyzed cell population is identified by the expression of one or more surface markers. In some instances, the one or more surface markers are cell type markers comprising CD45, CD14, CD3, CD4, CD8, CD19, CD11b, CD114, CD15, CD24, CD182, CD11a, CD91, CD16, CD25, Foxp3, CD20, CD38, CD22, CD61, CD56, CD31, CD30, CD38, CD62L, CD127, CD132, CD45RA, CD45RO, CD34, CD31, CD117, CD44, or a combination thereof. In some instances, the analyzed cell population comprises total lymphocytes that are positive for expression of the surface marker CD45. In some instances, the analyzed cell population comprises T lymphocytes that are positive for expression of the surface marker CD3. In some instances, the analyzed cell population comprises T helper/inducer lymphocytes that are positive for the expression of the surface marker CD4. In some instances, the analyzed cell population comprises T suppressor/cytotoxic lymphocytes that are positive for the expression of the surface marker CD8. In some instances, the analyzed cell population comprises monocytes that are positive for the expression of the surface marker CD14, CD11a, CD91, CD16, CD114, CD11b, or a combination thereof. In some instances, the analyzed cell population comprises monocytes that are positive for the expression of the surface marker CD14. In some instances, the analyzed cell population comprises B lymphocytes that are positive for the expression of the surface marker CD19. In some instances, the analyzed cell population comprises B lymphocytes that are positive for the expression of the surface marker CD20+, CD24+, CD38, CD22, or a combination thereof. In some instances, the analyzed cell population comprises granulocytes that are positive for the expression of the surface marker CD15+, CD182+, CD11b, CD24+, CD114+, or a combination thereof. In some instances, the analyzed cell population comprises granulocytes that are positive for the expression of the surface marker CD15+, CD182+, or a combination thereof. In some instances, the analyzed cell population comprises T regulatory cells that are positive for the expression of the surface marker CD4, CD25, Foxp3, or a combination thereof. In some instances, the analyzed cell population comprises natural killer cells that are positive for the expression of the surface marker CD56, CD31, CD30, CD38, or a combination thereof. In some instances, the analyzed cell population comprises thrombocytes that are positive for the expression of the surface marker CD61. In some instances, the analyzed cell population comprises stem cells that are positive for the expression of the surface marker CD34, CD117, or a combination thereof. In some instances, the analyzed cell population comprises naïve T lymphocytes that are positive for the expression of the surface marker CD45RA, CD127, CD132, CD62L, or a combination thereof. In some instances, the analyzed cell population comprises memory T lymphocytes that are positive for the expression of the surface marker CD45RO.

In some instances, an SI1 an SI2 or both are determined based on the expression level of CD69 in one or more analyzed cell populations selected from the list comprising total lymphocytes, T-lymphocytes, T helper/inducer lymphocytes, T suppressor/cytotoxic lymphocytes, monocytes, B lymphocytes, granulocytes, T regulatory cells, natural killer cells, thrombocytes, stem cells, naïve T lymphocytes, and memory T lymphocytes.

In some instances, an SI1 an SI2 or both are determined, individually, based on the expression level of CD69 in each of one or more analyzed cell populations selected from the list comprising total lymphocytes, T-lymphocytes, T helper/inducer lymphocytes, T suppressor/cytotoxic lymphocytes, monocytes, B lymphocytes, granulocytes, T regulatory cells, natural killer cells, thrombocytes, stem cells, naïve T lymphocytes, and memory T lymphocytes.

In some instances, an SI1 an SI2 or both are determined, individually, based on the expression level of CD69 in each of two, three, four, five, six, seven, eight, nine, ten, eleven or twelve analyzed cell populations selected from the list comprising total lymphocytes, T-lymphocytes, T helper/inducer lymphocytes, T suppressor/cytotoxic lymphocytes, monocytes, B lymphocytes, granulocytes, T regulatory cells, natural killer cells, thrombocytes, stem cells, naïve T lymphocytes, and memory T lymphocytes.

In some instances, an SI1 an SI2 or both are determined based on the expression level of CD28 in each of one or more analyzed cell populations selected from the list comprising total lymphocytes, T-lymphocytes, T helper/inducer lymphocytes, T suppressor/cytotoxic lymphocytes, monocytes, B lymphocytes, granulocytes, T regulatory cells, natural killer cells, thrombocytes, stem cells, naïve T lymphocytes, and memory T lymphocytes.

In some instances, an SI1 an SI2 or both are determined based on the expression level of CD28 in each of two, three, four, five, six, seven, eight, nine, ten, eleven or twelve analyzed cell populations selected from the list comprising total lymphocytes, T-lymphocytes, T helper/inducer lymphocytes, T suppressor/cytotoxic lymphocytes, monocytes, B lymphocytes, granulocytes, T regulatory cells, natural killer cells, thrombocytes, stem cells, naïve T lymphocytes, and memory T lymphocytes.

In some instances, relating comprises comparing one stimulation index to a univariate model that differentiates between two clinical categories. In some instances, relating comprises comparing the one or more stimulation indices to a multivariate model that differentiates between two clinical categories.

In some instances, the two clinical categories are chronic traumatic encephalopathy and healthy control, chronic traumatic encephalopathy and Alzheimer's Disease, chronic traumatic encephalopathy and Parkinson's disease, chronic traumatic encephalopathy and neural disorders that are not chronic traumatic encephalopathy, or chronic traumatic encephalopathy and not chronic traumatic encephalopathy. In some instances, the two clinical categories are chronic traumatic encephalopathy and healthy control. In some instances, the two clinical categories are chronic traumatic encephalopathy and Alzheimer's Disease. In some instances, the two clinical categories are chronic traumatic encephalopathy and Parkinson's disease. In some instances, the two clinical categories are chronic traumatic encephalopathy and neural disorders that are not chronic traumatic encephalopathy. In some instances, the two clinical categories are chronic traumatic encephalopathy and not chronic traumatic encephalopathy.

In some instances, the univariate model or multivariate model is capable of differentiating the two clinical categories with at least a 70%, 75%, 80%, 85%, 90%, 91%, 92%, 93%, 94%, 95%, 96%, 97%, 98%, or 99% positive agreement. In some instances, the univariate model or multivariate model is capable of differentiating the two clinical categories with at least a 70%, 75%, 80%, 85%, 90%, 91%, 92%, 93%, 94%, 95%, 96%, 97%, 98%, or 99% negative agreement.

In some instances, the subject is diagnosed with chronic traumatic encephalopathy based upon the relating.

In some instances, the subject is determined to have an increased risk of developing chronic traumatic encephalopathy based upon the relating.

In some instances, the subject is determined to have progressed to a more severe form of chronic traumatic encephalopathy based on the relating.

In some instances, the method further comprises quantifying the ratio of memory T lymphocytes to naïve T lymphocytes by quantifying the expression level of CD45RO and CD45RA and determining a change in CD45RO/RA ratio between the stimulated sample and the unstimulated sample.

In some instances, the method further comprises administration of MANF or a MANF peptidergic molecule that crosses the blood brain barrier to the subject that is diagnosed with chronic traumatic encephalopathy.

In some instances, the method further comprises administration of CDNF or a CDNF peptidergic molecule that crosses the blood brain barrier to the subject that is diagnosed with chronic traumatic encephalopathy.

Combinations with Other Diagnostic Modalities

The following tests can be used alone or conjunction with tests for understanding the proliferative state of the neural cells.

### Cerebrospinal Fluid (CSF)

CSF samples and protein assays of particular analytes can be used for assessing CTE. The procedure involves a lumbar puncture—the insertion of a hallow cannula or needle into the lower spinal column in order to collect 5-10 ml of CSF free of blood. In some embodiments neural disorders associated with trauma are assessed using either Saladax Biomedical/Ortho Clinical Diagnostics or Roche Diagnostics CSF Aβ42 and CSF Tau assays.

### Positron Emission Tomography (PET)

FDG-PET is an FDA approved tracer which measures glucose metabolism and has been successfully used to image brain energy consumption and is used as a radiotracer to in vivo label the amyloid plaques of the brain. In some embodiments these measurements are used to assess neural disorders associated with trauma.

### Magneto Encephalography (MEG)

These instruments employ advanced superconducting magnets near absolute zero temperatures to measure minute currents of the brain. They are fantastic instruments of technology but are scarcely available in the US, let alone other countries in the world. In some embodiments MEG is used to assess neural disorders associated with trauma.

### Magnetic Resonance Imaging (MRI)

These instruments are able to measure the gross anatomy of the brain within the skull with resolution approaching 100 microns in a standard 1.5 Tesla clinical MM. Although they are costly and accessible only at an imaging center (in patient or outpatient), they are standard of care to insure that there is no gross brain tumor or evidence of white matter infarct, typical after sub-clinical or mini strokes have occurred. In one modality, functional MRI is conducted whereby a patient is given tasks to complete while they are lying in a MRI brain scanner and asked to participate in task based maneuvers to understand which anatomical structures are active during which dynamic task. In some embodiments MRI is used to asses neural disorders associated with trauma.

### Electro Encephalography (EEG)

EEG is well known now for nearly a century since Hans Burger in 1928 discovered the surface potentials on the scalp. In contrast to most other neuro imaging techniques, EEG is trying to make movies of the brain to capture dynamics, not take static snapshots with long periodicity between them. In some embodiments EEG is used to assess neural disorders associated with trauma.

### Cognition

There are many companies creating cognitive assessments of a human subject from a neuropsychological perspective. Many of these are quite good, including the CogState battery of tasks, the CNS Vital Signs, and the CANTAB battery. This said, cognitive function relies on the integrated activity of many neuronal structures and processes and this may obscure early detection of underlying neuronal pathology. In addition, computer cognition assessment tools have limitations on their ability to accurately and objectively measure brain function. Equally importantly, they can be prone to subject bias as they require cooperation from the participant and can be fooled by human subjects interested to cheat the test/system. In some embodiments these measurements are used to assess neural disorders associated with trauma.

### Other Blood Tests

In some embodiments, other blood tests designed for Alzheimer's Disease are used to assess neural disorders associated with trauma. Today, there are few choices among blood tests for Alzheimer's disease. Many researchers and clinicians assay blood samples through the Myriad RBM multi-plexed platform where several pools of analytes are measured on a multiplexed Luminex analyzer providing information on hundreds of different analytes from a single drop of blood. Although the original panel was 89 analytes, it then grew to over 120, and is now approaching 250 such analytes. DiaGenic ASA (Oslo, Norway) has a novel blood test for the early detection of Alzheimer's disease based on a 96-gene expression array using an extracted RNA from blood based approach. Opko (Miami, Fla.) is using an antibody based method to fish out antigens and antibodies that are specific for a given condition from humans. They have published on their discovery of three peptoids that bind two different AD-specific antibodies and concluded a licensing deal with LabCorp in 2012. There appears to be a direct relationship between Opko's measured biomarkers and the publically available data. Cytox Ltd, a cell cycle dysfunction company, is focusing on measuring the G0 to G1 transition or G1/S cell cycle checkpoint.

Business Methods

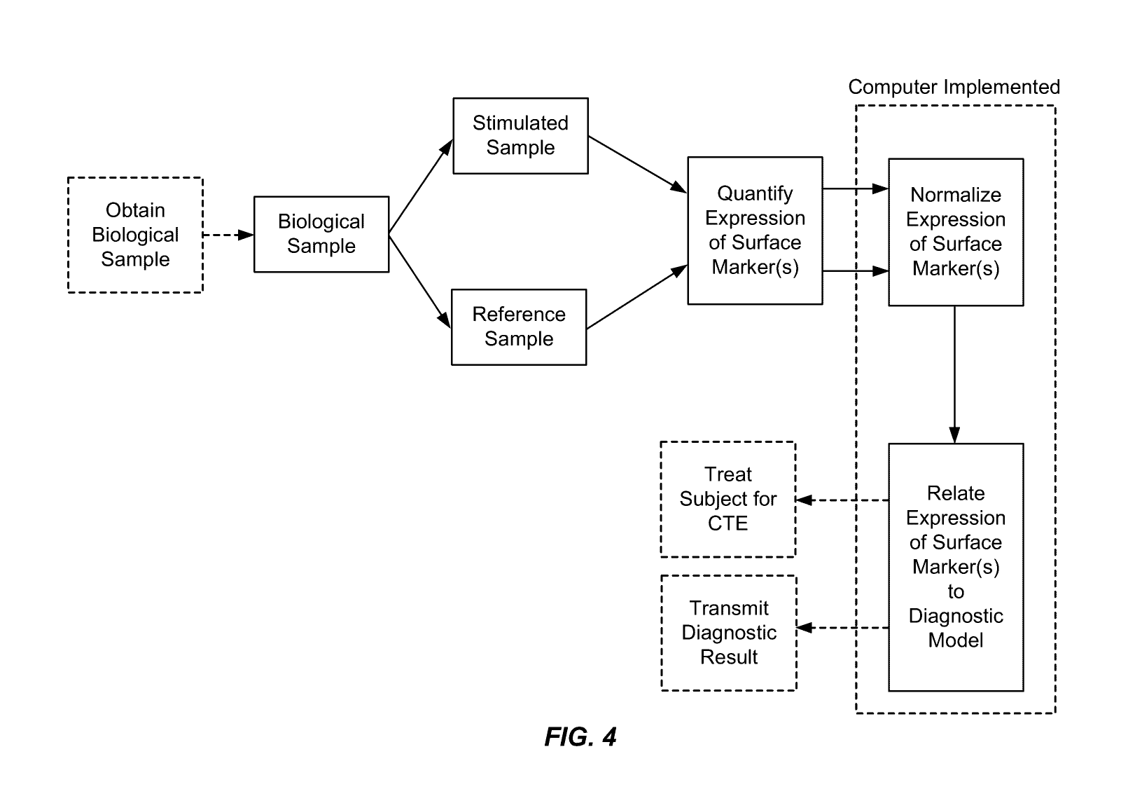

One or more computers may be utilized in the diagnostic methods disclosed herein, such as a computer 800 as illustrated in FIG. 4. The computer 800 may be used for managing subject and sample information such as sample or subject tracking, database management, analyzing cell surface marker data, determining one or more stimulation indices analyzing cytological data, storing data, billing, marketing, reporting results, or storing results. The computer may include a monitor 807 or other graphical interface for displaying data, results, billing information, marketing information (e.g. demographics), subject information, or sample information. The computer may also include means for data or information input 816, 815. The computer may include a processing unit 801 and fixed 803 or removable 811 media or a combination thereof. The computer may be accessed by a user in physical proximity to the computer, for example via a keyboard and/or mouse, or by a user 822 that does not necessarily have access to the physical computer through a communication medium 805 such as a modern, an internet connection, a telephone connection, or a wired or wireless communication signal carrier wave. In some cases, the computer may be connected to a server 809 or other communication device for relaying information from a user to the computer or from the computer to a user. In some cases, the user may store data or information obtained from the computer through a communication medium 805 on media, such as removable media 812. It is envisioned that data or diagnoses can be transmitted over such networks or connections for reception and/or review by a party. The receiving party can be, but is not limited to, an individual, a health care provider, or a health care manager. In one embodiment, a computer-readable medium includes a medium suitable for transmission of a result of an analysis of a biological sample, such as a level of one or more cell surface markers or one or more stimulation indices. The medium can include a result regarding a diagnosis of having, or being susceptible to, chronic traumatic encephalopathy for a subject, wherein such a result is derived using the methods described herein.

Sample information can be entered into a database for the purpose of one or more of the following: inventory tracking, assay result tracking, order tracking, subject management, subject service, billing, and sales. Sample information may include, but is not limited to: subject name, unique subject identification, subject-associated medical professional, indicated assay or assays, assay results, adequacy status, indicated adequacy tests, medical history of the subject, preliminary diagnosis, suspected diagnosis, sample history, insurance provider, medical provider, third party testing center or any information suitable for storage in a database. Sample history may include but is not limited to: age of the sample, type of sample, method of acquisition, method of storage, or method of transport.

The database may be accessible by a subject, medical professional, insurance provider, third party, or any individual or entity granted access. Database access may take the form of electronic communication such as a computer or telephone. The database may be accessed through an intermediary such as a customer service representative, business representative, consultant, independent testing center, or medical professional. The availability or degree of database access or sample information, such as assay results, may change upon payment of a fee for products and services rendered or to be rendered. The degree of database access or sample information may be restricted to comply with generally accepted or legal requirements for patient or subject confidentiality.

### EXAMPLES

### Example 1

A comparison of the Stimulation Index (SI) between the average of the control subjects to the average SI from the CTE subjects will be performed to distinguish the CTE subjects. The two sub-populations of lymphocytes will be compared. In this case, the CD4+ sub-population will be shown as the left pair on a graph, where CD4+ will indicates that the lymphocyte is a helper/inducer lymphocyte whereas the pair of bars on the right will reflect CD19+ lymphocytes, or those from B-cells which are involved in the humoral or antibody immune response. These experiments will indicate that the cells from the CTE population have impairment of mitogenic activation.

### Example 2

Once the analytical performance is established and stabilized over time, samples will be run on healthy control subjects and panels of human quality control samples. Using a small pilot set of CTE derived blood samples, analytic performance parameters will be evaluated, including estimates of Stimulation Index variance and SI effect size between CTE and control subjects in the newly established assay. This information will be vital to enable meaningful sample size N and power analysis to properly design clinical studies.

### Example 3

Blood from many CTE patients and healthy control subjects will be drawn and to each subject's blood, cells of interest (e.g. lymphocytes) will be purified, exposed to various stimulants which will trigger the cells to begin the cell division or proliferation process. By looking a half-day later at the cells, the expression of a cell surface marker, CD69, known to be reflective of cell cycle initiation and proliferation will be measured. An index for each human subject in the study as the ratio of the CD69 expression level when stimulated by mitogen divided by the endogenous CD69 level, a so-called “stimulation index” (SI), will be calculated. Then, the SI on various sub-populations of blood cells will be measured. It will be demonstrated that those with CTE will not able to enter the cell cycle as readily as the healthy control subjects. Without being bound by theory it is hypothesized that the dysfunction of the cellular regulatory machinery observed in the lymphocytes of the blood as measured by the “stimulation index,” will reflect the cellular machinery in the neurons of the brain responsible for the neuro degeneration in the CTE patients.

### Example 4

### Exemplary Test Protocol

Specimen Requirements

Whole blood specimens are received, for example, in a 10 mL CPT sodium heparin vacutainer (BD Vacutainer® CPT™ Cell Preparation Tube with Sodium Heparin. For the Separation of Mononuclear Cells from Whole Blood) filled about to capacity. Specimens are stored, shipped, or transported, as necessary, at room temperature (e.g., about 16-29° C.) until initial processing. Peripheral blood mononuclear cells (PBMCs) are cultured within 24 hours of collection from a subject.

PBMC Isolation from CPT and Culture Set Up

The CPT tube containing the whole blood specimen is centrifuged for about 20-30 minutes at 1500-1800 RCF (2800-3000 RPM). This separates the whole blood into a plasma layer (top), a mononuclear cell and platelet layer (middle) and a red blood cell layer (bottom). The mononuclear cell and platelet layer and the red blood cell layer are separated by a polymer gel. The plasma layer is aspirated and discarded (generally to within about a ½ inch of the mononuclear cell and platelet layer. 5 mL of room temperature RPMI 1640 media, without L-Glutamine (HyClone Catalog # SH30096.02) are added to the tube, the mononuclear cell and platelet layer is gently resuspended, and then transferred to a 15 mL conical tube. 5 mL of RPMI media is used to wash the top and along the edges of the gel plug and transferred to the 15 mL conical tube. Multiple samples from the same subject can be combined at this stage, if necessary.

The 15 mL conical tube is centrifuged at about 400 RCF (1350-1450 rpm) for about 10 minutes and the supernatant is removed. Then the tube is gently vortexed to resuspend the pellet in about 10.5 mL of RMPI media. 200 μL of the cell suspension is removed to a 12×75 mm polystyrene tube, and cell count and viability are determined us a Nexcelom Auto 2000 Cellometer.

A calculation to determine the final volume is performed based upon the cell counts and % viability according to the following equation:

$$Final Volume = \frac{( \frac{\% Viability}{100} ) ( Cell Counts ( {10}^{6} \text{/} mL ) ) ( 10 mL )}{8.0 \times {10}^{6} \text{/} mL}$$

The tube containing the bulk of the cells is centrifuged at about 400 RCF (1350-1450 rpm) for about 10 minutes and the supernatant is removed. The cell pellet is gently resuspended in the calculated Final Volume of complete media (RPMI 1640+10% FBS+1% L-Glutamine and 1% Pen/Strep; BD 90M047) to produce an 8.0×10⁶ cells/mL suspension.

Three conditions are prepared: (1) unstimulated, (2) phytohemagglutinin (PHA-L) stimulated, and (3) pokeweed mitogen (PWM) stimulated in separate 15 mL conical tubes. 380 μL of PBMC cells at 80×10⁶ cells/mL are added to each tube. Then 20 μL of complete media is added to the unstimulated control; 20 μL of PHA-L (240 μg/mL) is added to the PHA-L stimulated condition; and 20 μL of PWM (80 μg/mL) is added to the PWM stimulated condition. The tubes are mixed gently and placed in a 20° slant rack with loosened caps and then incubated for about 4 hours (±10 minutes) in an incubator at about 37° C. (+/−2° C.) and about 5% CO₂ (±2%).

Post Stimulation Cell Storage

After stimulation, the cells are washed by adding about 10 mL of Wash Buffer (1×PBS, 0.09% NaN3, 1% FBS; BD 90B050), centrifugation at about 400 RCF (1350-1450 rpm) for about 10 minutes, and removal of all but about 50-100 μL of the supernatant. The cells are gently resuspended in the remaining supernatant, 1 mL of Freezing Buffer (90% FBS, 10% DMSO) is added to each tube followed by gentle mixing, and the tubes are placed at about −70° C. to about −85° C. for storage.

Staining

Fresh Working Benzonase Solution is prepared by adding 30 μL of Novagen Stock Benzonase Solution (70746-3) per 10 mL of complete media (RPMI 1640+10% FBS+1% L-Glutamine and 1% Pen/Strep; BD 90M047). For each subject, the frozen cells from all three conditions (unstimulated, PHA-L stimulated, and PWM stimulated) are thawed at about 37° C. (±2° C.) in a water bath. The tubes are removed from the water bath as the last ice crystals are observed.

2 mL of Working Benzonase solution is added to each tube, followed by gentle mixing, then 10 mL of complete media is added to each tube. The tubes are then mixed by inversion. The tubes are then centrifuged at about 400 RCF (1350-1450 rpm) for about 10 minutes, and all but about 50 μL of the supernatant is removed, without disturbing the cell pellet. The pellet is gently vortexed to resuspend, 10 mL of Wash Buffer (1×PBS, 0.09% NaN3, 1% FBS; BD 90B050) is added, and the suspension is gently mixed. The tubes are then centrifuged at about 400 RCF (1350-1450 rpm) for about 10 minutes, and all but about 50 μL of the supernatant is removed, without disturbing the cell pellet. The pellet is gently vortexed to resupend, about 200 μL of Wash Buffer is added, and the suspension is gently mixed.

Each condition is split into two tubes (12×75 polystyrene) containing about 100 μL of cell suspension, for a total of six tubes. To each tube, 20 μL of an antibody cocktail is added. Cocktail 1 (“isotype cocktail”), which is added to one tube in each condition, contains α-CD3-FITC, α-IGG1-PE, α-CD14-PerCP-Cy5.5, α-CD4-PE-CY7, α-IGG1-APC, α-CD45-APC-H7, α-CD19-V450, and α-CD8-V500 antibodies. Cocktail 2 (“test cocktail”), which is added to the other tube in each condition, contains α-CD3-FITC, α-CD69-PE, α-CD14-PerCP-Cy5.5, α-CD4-PE-CY7, α-CD28-APC, α-CD45-APC-H7, α-CD19-V450, and α-CD8-V500 antibodies. Each cocktail contains a mixture of cell-type markers and activation markers. The cells are then incubated at room temperature (about 16-29° C.) for about 30 minutes, while being protected from light.

The cells are then washed. Briefly, the tubes are centrifuged at about 400 RCF (1350-1450 rpm) for about 5 minutes, and all but about 50 μL of the supernatant is removed, without disturbing the cell pellet; 2 mL of Wash Buffer is added to each tube; the tubes are centrifuged at about 400 RCF (1350-1450 rpm) for about 5 minutes, and all but about 50 μL of the supernatant is removed, without disturbing the cell pellet; and about 0.25 mL of Wash Buffer are added and the cells are gently resuspended. The stained cells can be stored for about 6-8 hours at about 2-8° C., if desired.

Comp Tube Preparation and Acquisition

For each tube of stained cells (6 tubes per subject, 2 per condition), nine (9) 12×75 polystyrene tubes are prepared—one negative control and one tube for each of eight labels. To each 12×75 polystyrene tube, 100 μL of Wash Buffer and about 60 μL of the appropriate BD CompBeads (BD 552843), and an appropriate amount (e.g., 20 μL, 10 μL, 5 μL, etc.) of each compensation reagent [CD45 FITC antibody (Cat#347463), CD3 PE antibody (Cat#347347), CD45 PerCP-CyTM5.5 antibody (Cat#340953), CD4 PE-CY7 Compensation reagent (batch match) (Cat# BP80449-03), CD45 APC antibody (Cat#340943), CD45 APC-H7 Compensation reagent (batch match) (Cat# BD80449-04), CD45 V450 antibody (Cat#560367), or CD45 V500 antibody (Cat#560777)] is added. The comp beads are incubated for about 15-30 minutes at room temperature, in the dark.

After incubation, the comp beads are washed as follows: about 2 mL of Wash Buffer is added to each tube, the tubes are centrifuged at about 200 RCF (about 1000 rpm) for about 5 minutes, all but about 50 μL of the supernatant is removed, and about 500 μL of Wash Buffer is added to resuspend the beads. The comp beads can be stored for about 2 hours at about 2-8° C. before use.

Flow Cytometry and Analysis

The samples are analyzed by flow cytometry, for example, using a Becton Dickinson FacsCanto II machine. A set number of gated events is collected for each condition. For example, about 1×10⁴ gated events can be collected for each condition. In another example, about 5×10⁴ CD45+ gated events can be collected for each condition.

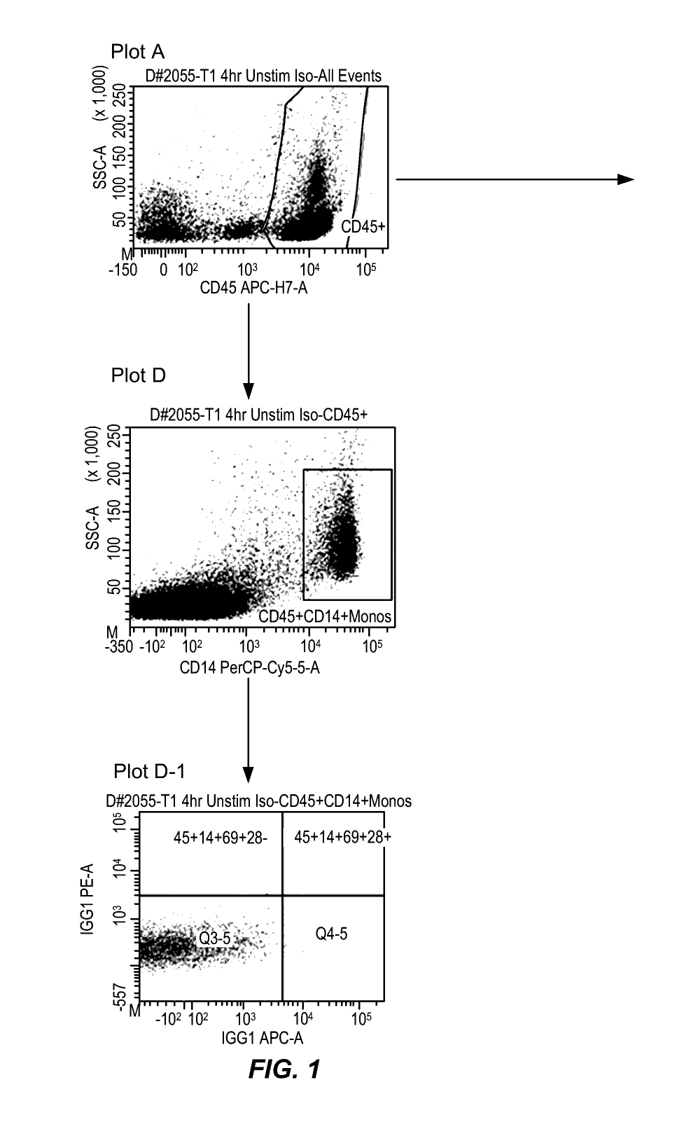

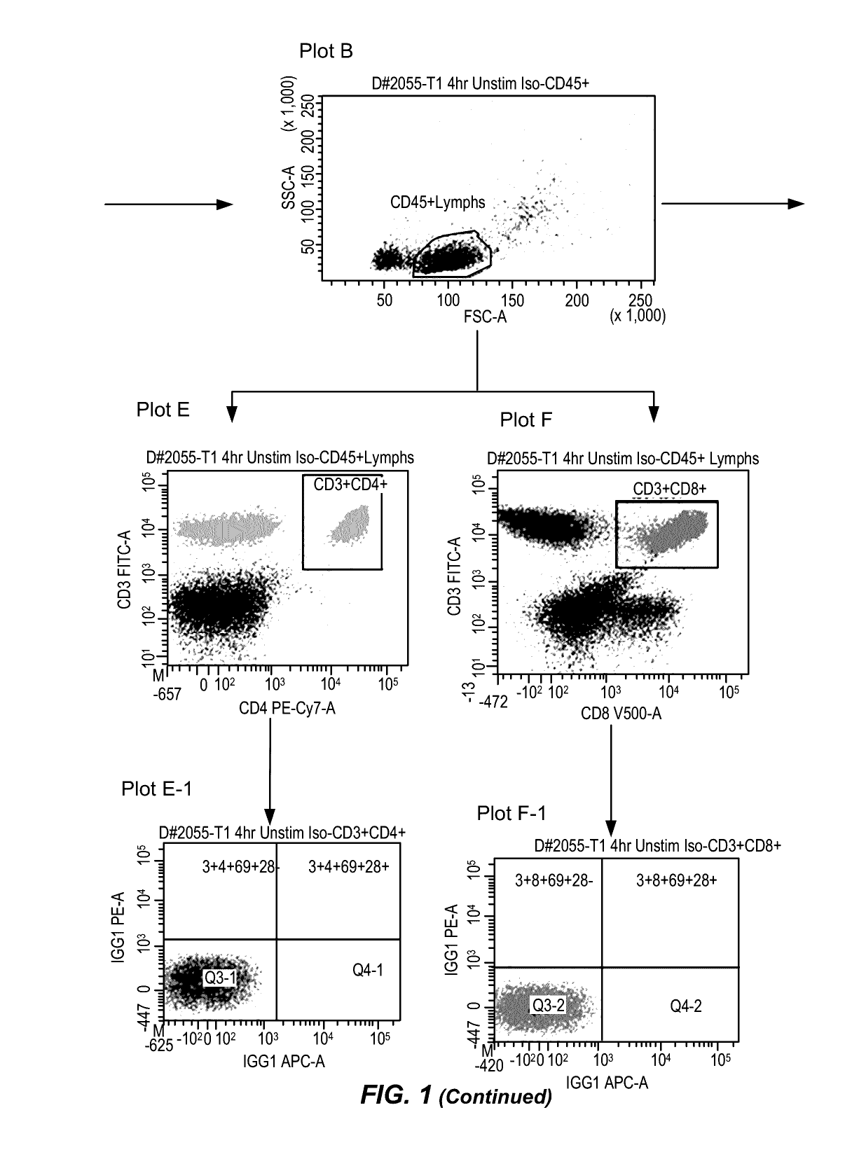

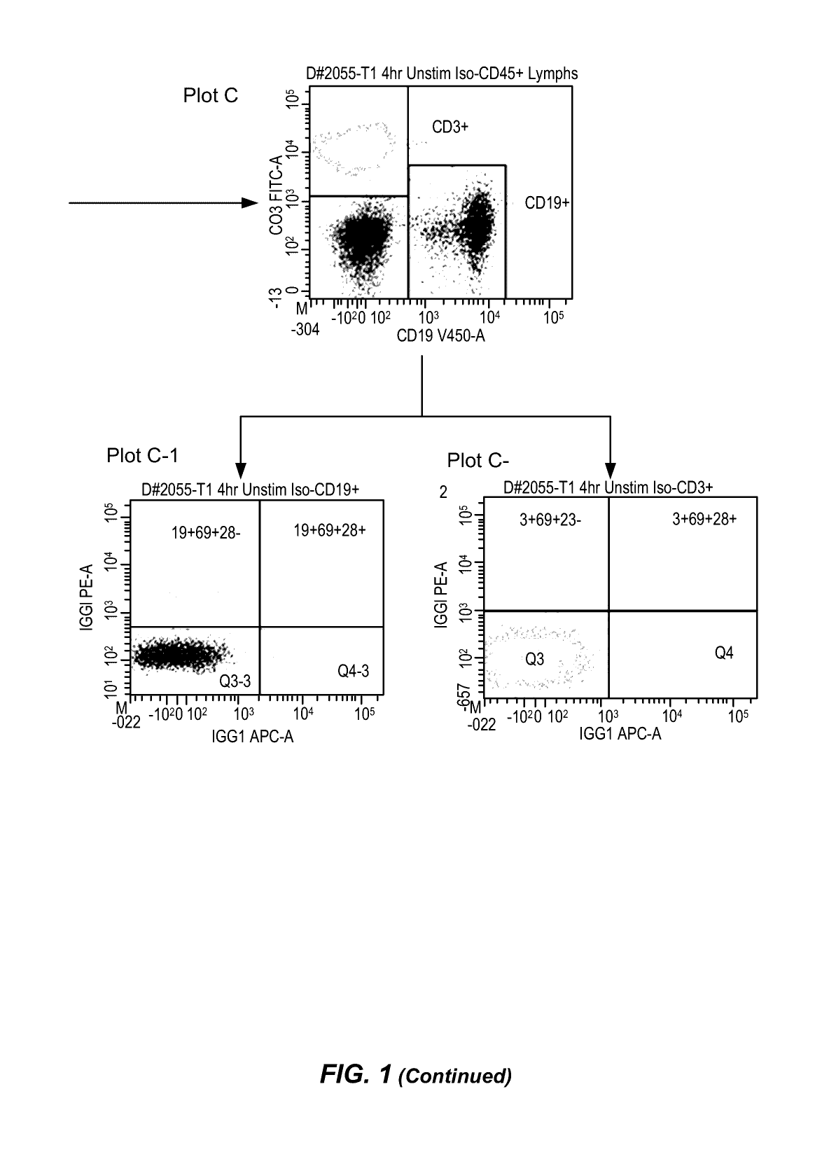

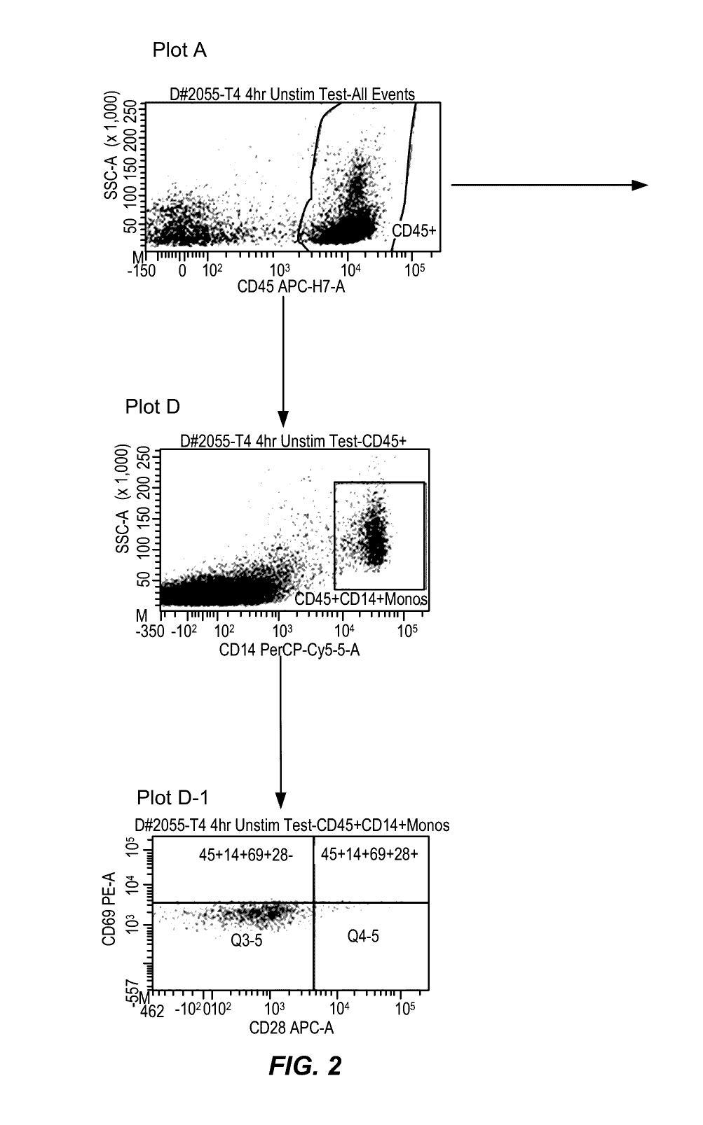

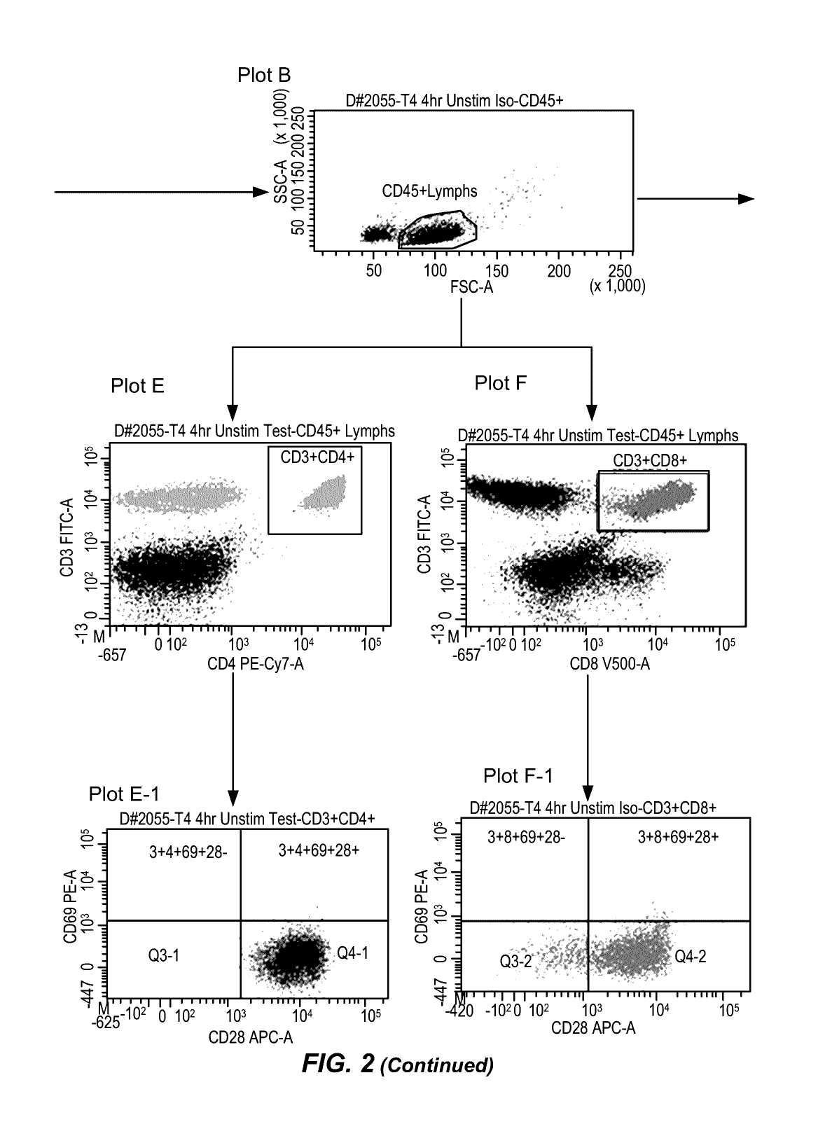

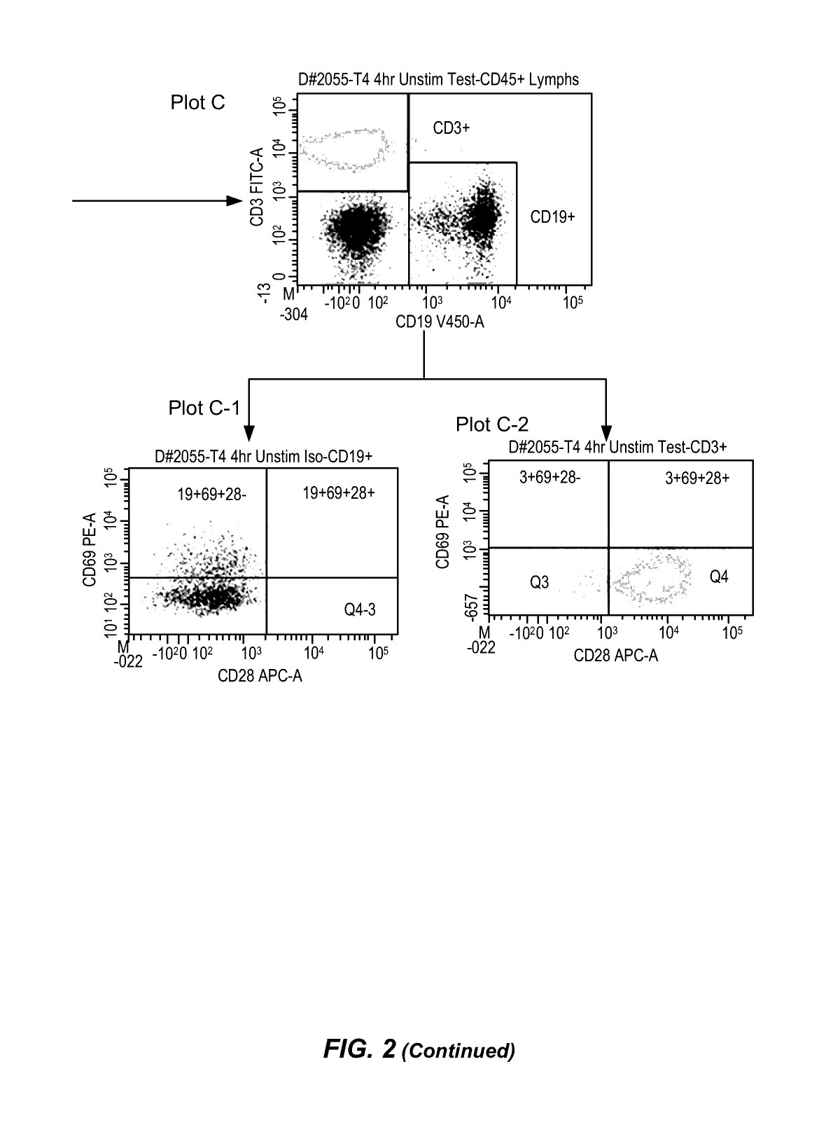

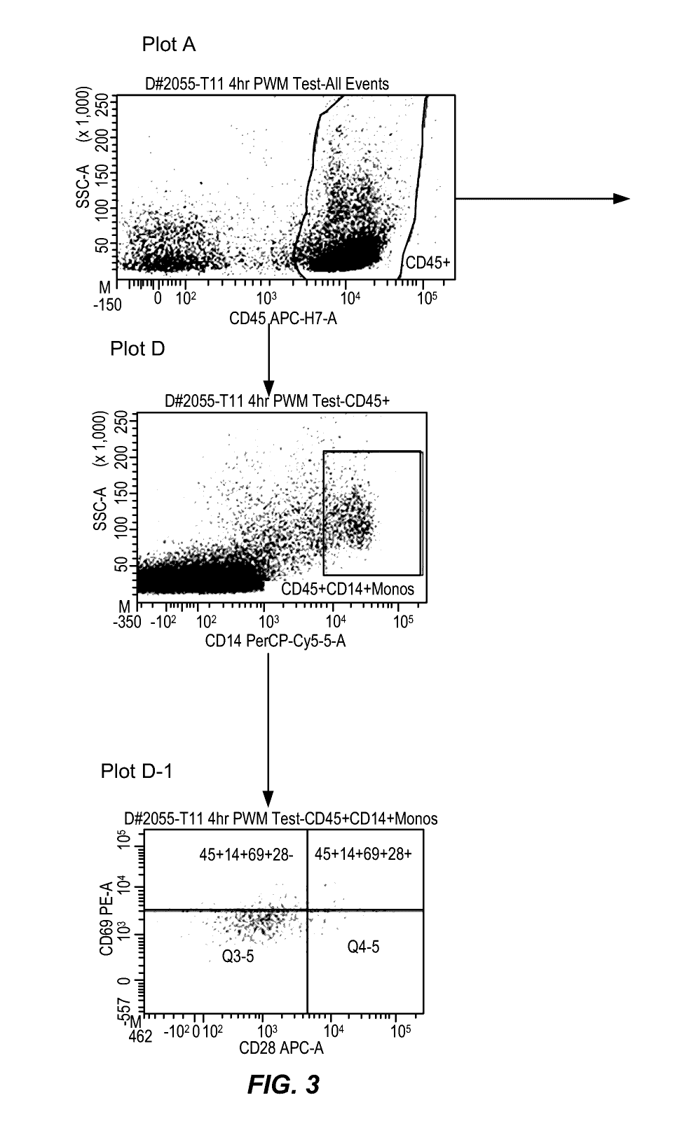

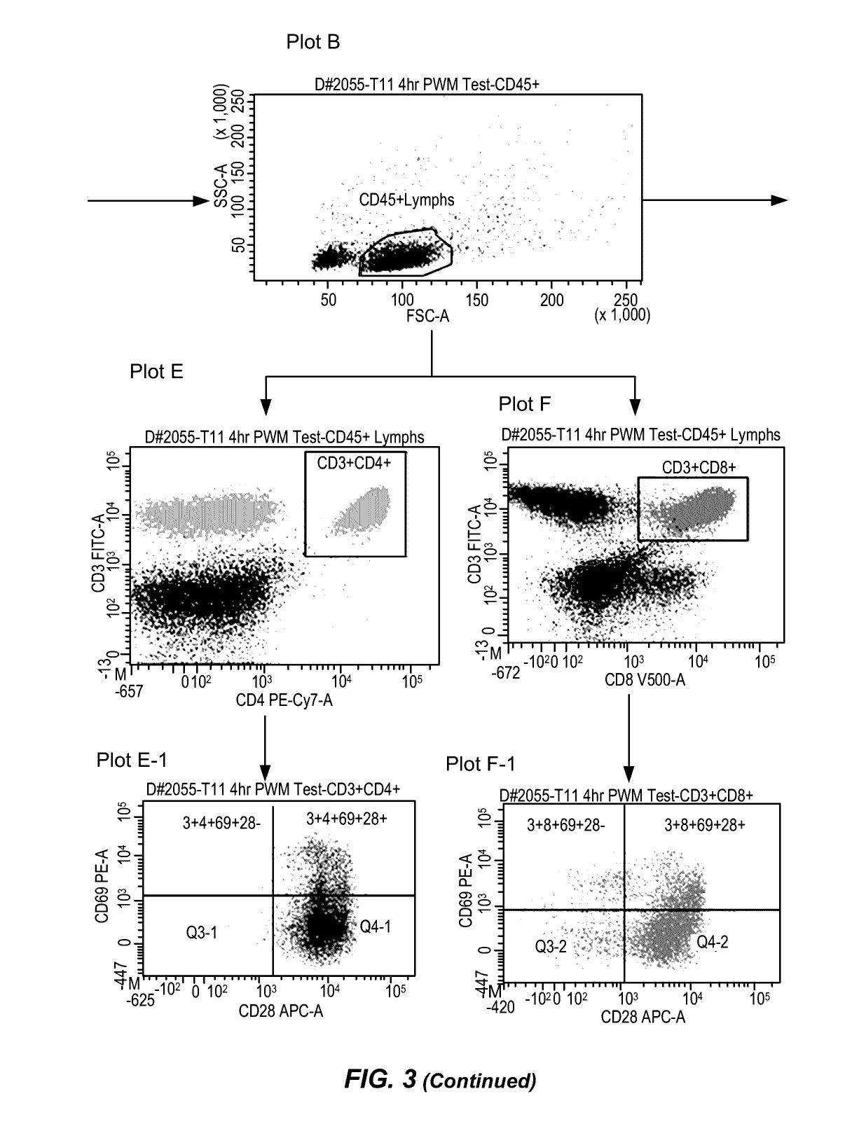

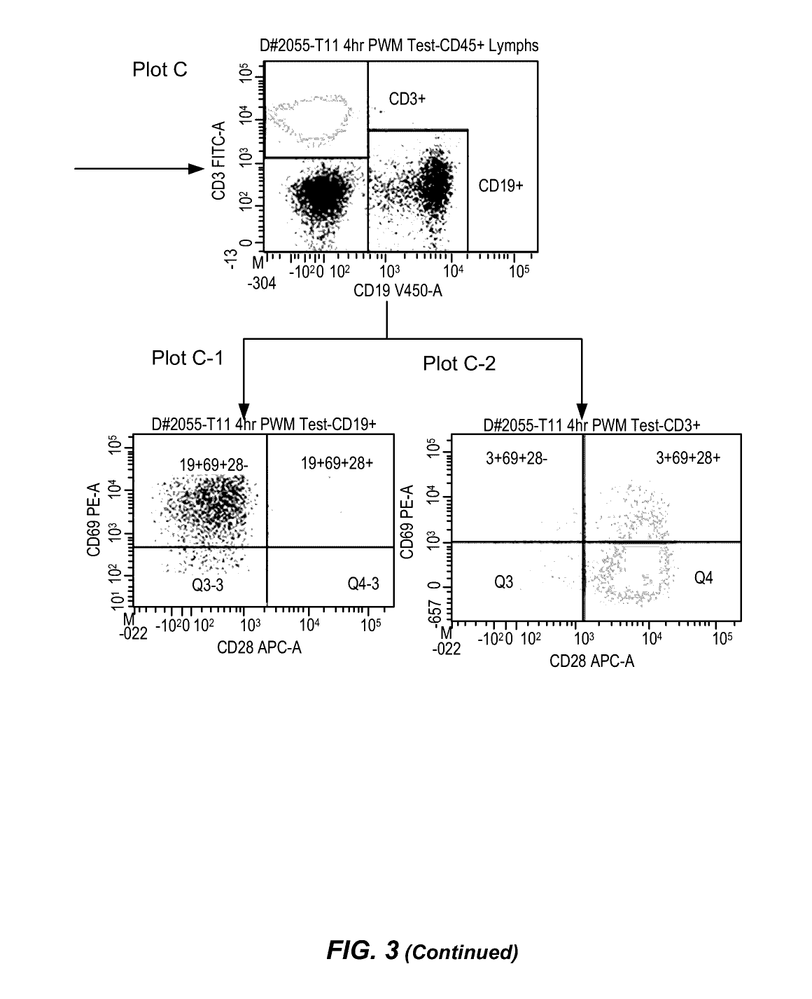

The flow cytometry data is analyzed using appropriate software such as BD FACSDiva software. Data from an unstimulated cell sample stained with the isotype cocktail are used to initially set the gates; for example as illustrated in FIG. 1. Data from an unstimulated cell sample stained with the test cocktail is used to verify the gate placements; for example, as illustrated in FIG. 2. Data from a stimulated cell sample stained with the test cocktail is used to further verify the gate placements; for example, as illustrated in FIG. 3. Then, all samples are analyzed according to the verified gate placements.

Flow cytometry data is self-normalized, where each subject's mitogenic response data from stimulated samples are adjusted to account for basal mitogenic activity as determined by the corresponding nonstimulated reference sample. Self-normalization can be accomplished by determining a stimulation index 1 (SI1) defined by the ratio of the percentages of cells positive for an activation marker, with and without mitogenic stimulation, within an analyzed cell population (e.g., within a population identified by a cell type marker). Self-normalization can also be accomplished by determining a stimulation index 2 (SI2) defined by the ratio of the mean activation marker expression within an analyzed cell population (e.g., within a population identified by a cell type marker) with and without mitogenic stimulation. The formulas for SD and SI2 are as follows:

$$SI 1 = \frac{( {Marker}^{+} Cells / Total Cells ) stim}{( {Marker}^{+} Cells / Total Cells ) unstim}$$

$$SI 2 = \frac{( Mean Marker Int . ) stim}{( Mean Marker Int . ) unstim}$$

### Example 5

### Exemplary Study to Develop Univariate and Multivariate Diagnostic Models

Participants

Subjects will be recruited within clinical categories that are well-matched for age, gender, severity of dementia (except for healthy controls), and most other characteristics. The clinical categories for recruitment will include subjects with a probable diagnosis of chronic traumatic encephalopathy and healthy controls. Clinical categories containing subjects diagnosed with, or with a probable diagnosis of, another neuropathy such as Alzheimer's Disease or Parkinson's Disease can also be recruited. The recruited subjects will be informed consented and will have been evaluated according to the mini-mental state examination (MMSE) to screen for degree of cognitive impairment. Complete medical histories can also be taken, or made available, for each subject.

Obtaining Biological Samples

A phlebotomist will obtain 56 mL of blood in total from each subject via venipuncture. 16 mL of the blood will be shipped to a clinical laboratory and tested according the procedures outlined in Example 4. The blood sample will be coded with the subject study ID and all testing will be performed without knowledge of the individual subject's clinical category.

Statistical Analysis Methods/Model Development

General Statistical Methods

“Descriptive statistics” refers to mean, median, standard deviation (SD), minimum and maximum for continuous measurements, and number and percentage of patients in each level of a categorical measurement. All exploratory statistical tests will be 2-tailed and performed at the 5% significance level, unless stated otherwise. Missing values will not be imputed for any variable.

Demographics and Baseline Clinical Covariates

Demographic variables, available baseline clinical covariates, medical histories, previous and concurrent medications, and Mini Mental State Examination (MMSE) scores will be analyzed descriptively for (a) the entire population of study patients, and individually by (b) the clinical category of the subjects.

Differences in distribution of clinical and demographic variables between the sub-populations will be analyzed using the non-parametric Wilcoxon Rank Sum test for continuous variables, and the Chi-square or Fisher's Exact tests for categorical variables as appropriate. Those variables displaying significant distributional differences between sub-populations may be included as predictive covariates in multivariate logistic regression and Receiver-Operating Characteristic (ROC) curve analyses.

Efficacy Analyses

Efficacy analyses will be applied in exploratory mode to all assay output parameters and variable expressions of interest. The following general methods will be employed.

Distributions of Assay Results

Descriptive statistics including mean, median, standard deviation, minimum, and maximum will be presented by clinical category (e.g., CTE or healthy control). A bivariate analysis and assessment of separation using the 2-sample Wilcoxon Rank Sum test will be performed.

2×2 Contingency Table and Estimates of Diagnostic Performance

Binary categorizations of patients based on assay results will be cross-referenced to their clinical category (e.g., CTE or healthy control) to construct 2×2 contingency tables. Sensitivity, specificity, positive predictive value (PPV), negative predictive value (NPV), positive likelihood ratio, and negative likelihood ratio will be estimated along with their associated 2-sided Wilson Score 95% Confidence Intervals (95% CIs).

Univariate and Multivariate Logistic Regression and ROC

Uni- and multivariate logistic regression models will be employed utilizing binary expressions of assay results versus the binary clinical category (e.g., CTE or healthy control). Regression coefficient, standard error (SE), odds ratio (OR) and its associated 95% CI, and Wald p-value will be reported for each term in a model.

A univariate receiver-operating characteristic (ROC) curve for association of asssay test results versus the binary clinical category (e.g., CTE or healthy control) will be constructed expressing assay results as continuous variables. Area under the curve (AUC) and its associated 95% CI will be reported for each analysis. Additionally, multivariate ROCs may be constructed utilizing the output of the logistic regression models. Comparison of ROCs will rely on the method of DeLong (DeLong E R, DeLong D M, Clarke-Pearson DL. Comparing the areas under two or more correlated receiver-operating characteristic curves: a nonparametric approach. Biometrics 1988; 44:837.)

Algorithmic Approaches to Maximize Separation of Clinical Categories

The markers making up the test will be analyzed separately in a backwards-selection logistic regression model employing the clinical categorization (e.g., CTE or healthy control) as the dependent variable. With each run of the model, the term with the highest Wald p-value will be discarded and the model re-run. The process will proceed iteratively until only terms with p<0.05 remain. The final model along with coefficients will be reported.

Other Analyses

Dementia severity will be expressed as categorical levels. Assay results expressed as continuous variables will be plotted (mean values) by categorical dementia level as a barplot. A test for statistical trend may be performed. Spearman's correlation analysis will also be performed to investigate the presence and degree of correlation between continuous assay results and MMSE score results (continuous). Lastly, distributions of assay test scores will be presented for sub-groups of patients receiving various drugs of interest.

Intra- and Inter-Individual Biological Variability (Substudy)

Intra-individual biological variation will be calculated for each patient in the substudy using the equation:

CV_{T}²=CV_{A}²+CV_{I}²

where CV_{T} is the total variation, CV_{A} is the intra-assay variation, and CV_{I} is the intra-individual biological variation (Sarno M, Powell H, Tjersland G, et al. A collection method and high-sensitivity enzyme immunoassay for sweat pyridinoline and deoxypyridinoline cross-links. Clin Chem 1999; 45:9.). Following calculation of CV_{I} for all patients in the sub-study, a 95% CI for the population will be calculated as: Mean±(S.E.*t_{95%}*sqrt (1+1/N)) where S.E.=standard error of the mean, and t_{95%} is the t-value at 95% confidence for the appropriate degrees of freedom (n−1).

Inter-individual variation (CV_{G}) will also be determined via calculation of the % CV from mean values for all patients.

As desired, the index of individuality will be calculated using the following equation: I.I.=CV_{I}/CV_{G}

An I.I. value exceeding 1.4 generally indicates that conventional reference intervals are informative.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1-72. (canceled)

73. A method, comprising:
(a) preparing a stimulated sample by culturing a first portion of a biological sample obtained from a subject with one or more of mitogenic compounds and a reference sample by culturing a second portion of the biological sample without the one or more mitogenic compounds;
(b) quantifying an expression of one or more surface markers in the stimulated sample and the reference sample;
(c) normalizing the expression of the one or more surface markers in the stimulated sample with the expression of the one or more surface markers in the reference sample by determining one or more stimulation indices; and
(d) relating the one or more stimulation indices to an assessment of the subject for a risk of developing chronic traumatic encephalopathy, a diagnosis of chronic traumatic encephalopathy, or a measurement of a progression of chronic traumatic encephalopathy.

74. The method of claim 73, wherein the biological sample comprises a tissue sample, a blood sample, a bone marrow sample, a cerebrospinal fluid sample, or any combination thereof.

75. The method of claim 74, wherein the biological sample comprises a blood sample.

76. The method of claim 75, wherein the stimulated sample and the reference sample are produced from peripheral blood mononuclear cells (PMBCs) from the blood sample.

77. The method of claim 73, wherein the one or more mitogenic compounds comprise phytohaemagglutinin (PHA-L), pokeweed mitogen (PWM), or a combination thereof.

78. The method of claim 73, wherein the quantifying comprises staining the stimulated sample and the reference sample with one or more antibodies that specifically bind the one or more surface markers.

79. The method of claim 73, wherein the quantifying comprises fluorescent activated cell sorting, western blotting, ELISA analysis, magnetic cell sorting, or any combination thereof.

80. The method of claim 73, wherein the one or more surface markers comprise CD69, CD28, CD45, CD14, CD3, CD4, CD8, CD19, CD11b, CD114, CD15, CD24, CD182, CD11a, CD91, CD16, CD25, Foxp3, CD20, CD38, CD22, CD61, CD56, CD31, CD30, CD38, CD62L, CD127, CD132, CD45RA, CD45RO, CD34, CD31, CD117, CD44, or any combination thereof.

81. The method of claim 73, wherein the one or more surface markers comprise CD45, CD14, CD3, CD4, CD8, CD19, CD69, CD28, or any combination thereof.

82. The method of claim 73, wherein the one or more surface markers comprise CD45, CD14, CD3, CD4, CD8, CD19, CD69, and CD28.

83. The method of claim 73, wherein the normalizing is computer implemented.

84. The method of claim 73, wherein the one or more stimulation indices comprise a stimulation index 1 (SI1) defined by a ratio of a percentage of cells positive for one of the one or more surface markers with and without mitogenic stimulation within an analyzed cell population.

85. The method of claim 73, wherein the one or more stimulation indices comprise a stimulation index 2 (SI2) defined by a ratio of mean expression for one of the one or more surface markers within an analyzed cell population with and without mitogenic stimulation.

86. The method of claim 73, wherein the relating is computer implemented.

87. The method of claim 73, wherein relating comprises comparing one stimulation index to a univariate or multivariate model that differentiates between two clinical categories.

88. The method of claim 87, wherein the univariate model or multivariate model is capable of differentiating the two clinical categories with at least a 70% positive or negative agreement.

89. The method of claim 73, wherein the subject is diagnosed with chronic traumatic encephalopathy based upon the relating.

90. The method of claim 73, wherein the subject is determined to have an increased risk of developing chronic traumatic encephalopathy based upon the relating.

91. The method of claim 73, wherein the subject is determined to have progressed to a more severe form of chronic traumatic encephalopathy, based upon the relating.

92. A method of diagnosing a subject with chronic traumatic encephalopathy, an early-stage of chronic traumatic encephalopathy, or a predisposition for chronic traumatic encephalopathy, the method comprising:
(a) isolating peripheral blood mononuclear cells (PMBCs) from a blood sample obtained from the subject;
(b) culturing a first portion of the PMBCs with one or more mitogenic compounds to produce a stimulated sample;
(c) culturing a second portion of the PMBCs without the one or more mitogenic compounds to produce a reference sample;
(d) quantifying an expression of one or more surface markers in the stimulated sample and the reference sample;
(e) normalizing the expression of the one or more surface markers in the stimulated sample with the expression of the one or more surface markers in the reference sample by determining one or more stimulation indices; and
(f) relating the one or more stimulation indices to an assessment of a risk of developing chronic traumatic encephalopathy, a diagnosis of chronic traumatic encephalopathy, or a measurement of a progression of chronic traumatic encephalopathy.
